SEQUENCE LISTING (13 sequences)

SEQ ID NO: 1 - DNA, 29903 bp, SARS-CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 1260 bp, SARS-CoV-2
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 3 - DNA, 72 bp, SARS-CoV-2
gaccccaaaatcagcgaaatgcaccccgcattacgtttggtggaccctcagattcaactggcagtaaccaga

SEQ ID NO: 4 - DNA, 67 bp, SARS-CoV-2
ttacaaacattggccgcaaattgcacaatttgcccccagcgcttcagcgttcttcggaatgtcgcgc

SEQ ID NO: 5 - DNA, 20 bp, Artificial Sequence
  Primer
gaccccaaaatcagcgaaat

SEQ ID NO: 6 - DNA, 24 bp, Artificial Sequence
  Primer
tctggttactgccagttgaatctg

SEQ ID NO: 7 - DNA, 24 bp, Artificial Sequence
  Detection Probe
accccgcattacgtttggtggacc

SEQ ID NO: 8 - DNA, 22 bp, Artificial Sequence
  Primer
gggagccttgaatacaccaaaa

SEQ ID NO: 9 - DNA, 21 bp, Artificial Sequence
  Primer
tgtagcacgattgcagcattg

SEQ ID NO: 10 - DNA, 24 bp, Artificial Sequence
  Detection Probe
atcacattggcacccgcaatcctg

SEQ ID NO: 11 - DNA, 19 bp, Artificial Sequence
  Primer
agatttggacctgcgagcg

SEQ ID NO: 12 - DNA, 20 bp, Artificial Sequence
  Primer
gagcggctgtctccacaagt

SEQ ID NO: 13 - DNA, 23 bp, Artificial Sequence
  Detection Probe
ttctgacctgaaggctctgcgcg

(19) United States
(12) Patent Application Publication
(10) Pub. No.: US 2021/0108259 A1
(43) Pub. Date: Apr. 15, 2021

(54) COMPOSITIONS, METHODS AND KITS FOR BIOLOGICAL SAMPLE AND RNA STABILIZATION

(71) Applicant: Liquid Biopsy Research LLC, Charlestown (KN)

(72) Inventors: Mark KIDD, New Haven, CT (US); Irvin Mark MODLIN, Woodbridge, CT (US); Ignat DROZDOV, Stratford Upon Avon (GB)

(73) Assignees: Liquid Biopsy Research LLC, Charlestown (KN); Liquid Biopsy Research LLC, Charlestown (KN)

(21) Appl. No.: 17/065,675

(22) Filed: Oct. 8, 2020

Related U.S. Application Data
(60) Provisional application No. 62/913,458, filed on Oct. 10, 2019.

(51) Int. Cl. C12Q 1/6848; C12Q 1/686

(52) U.S. Cl. CPC C12Q 1/6848; C12Q 1/686

(57) ABSTRACT

The present disclosure provides a buffer comprising at least one chaotropic agent, at least one chelating agent and at least one non-ionic surfactant, wherein the buffer stabilizes a biological sample at about room temperature for at least about one day.

### RELATED APPLICATIONS

This application claims priority to and the benefit of U.S. Provisional Application No. 62/913,458, filed Oct. 10, 2019, the content of which is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Oct. 6, 2020, is named “LBIO-006_SeqList.txt” and is about 42.1 KB in size.

### BACKGROUND

Biological samples, including blood samples and saliva samples, are known to degrade over time. More specifically, RNA within biological samples are known to degrade over time, with degradation especially problematic when the biological samples are either stored at room temperature for an extended period of time, or are subjected to a wide range of temperatures of a given amount of time (referred to as thermal excursions or temperature excursions). Thus, to preserve biological samples for use in subsequent diagnostic, treatment and research methods, specialized equipment and supplies are required, including snap-freezing reagents and freezers capable of maintaining samples at super-low temperatures such as −80° C. Furthermore, the freezing process is also known to damage biological samples and the RNA contained in those samples. Thus, there exists a need in the art for alternative compositions, methods and kits for stabilizing biological samples, including blood samples and saliva samples. Such stabilization compositions, methods and kits could be used in combination with a variety of different diagnostic, treatment and research methods, including, but not limited to methods for the diagnosis, prognosis, treatment and monitoring of Gastroenteropancreatic (GEP) neuroendocrine neoplasm (GEP-NEN) as well as methods of diagnosing viral infections, such as infection with SARS-CoV-2.

### SUMMARY

The present disclosure provides stabilization buffers comprising: (a) guanidine hydrochloride at a concentration of about 4.05 M to about 4.95 M; (b) Triton X-100 at a concentration of about 0.09% to about 0.11% (v/v); and (c) EDTA at a concentration of about 18 mM to about 22 mM, wherein the pH of the stabilization buffers is less than about 4.5. In some aspects, the stabilization buffers can comprise: (a) guanidine hydrochloride at a concentration of about 4.275 M to about 4.725 M; (b) Triton X-100 at a concentration of about 0.095% to about 0.105% (v/v); and (c) EDTA at a concentration of about 19 mM to about 21 mM. In some aspects, the stabilization buffers can comprise: (a) guanidine hydrochloride at a concentration of about 4.5 M; (b) Triton X-100 at a concentration of about 0.1% (volume/volume); and (c) EDTA at a concentration of about 20 mM. In some aspects, the pH of the stabilization buffers can be between about 4.05 and about 4.11.

In some aspects, the stabilization buffers of the present disclosure can further comprise: (d) citric acid at a concentration of about 72 mM to about 88 mM; and (e) sodium citrate at a concentration of about 108 mM to about 132 mM. In some aspects, the stabilization buffers can comprise: (d) citric acid at a concentration of about 76 mM to about 84 mM; and (e) sodium citrate at a concentration of about 114 mM to about 126 mM. In some aspects, the stabilization buffers can comprise: (d) citric acid at a concentration of 80 mM; and (e) sodium citrate at a concentration of 120 mM.

The present disclosure provides stabilization buffers; wherein the stabilization buffers comprise: (a) guanidine hydrochloride at a concentration of about 4.5 M; (b) Triton X-100 at a concentration of about 0.1% (volume/volume); (c) EDTA at a concentration of about 20 mM; (d) citric acid at a concentration of 80 mM; and (e) sodium citrate at a concentration of 120 mM, wherein the pH of the stabilization buffers is between about 4.05 and about 4.11.

The present disclosure provides compositions comprising a mixture of: (a) any one of the preceding stabilization buffer of any of the preceding claims; and (b) a biological sample isolated from a subject. In some aspects, the biological sample can comprise at least one RNA transcript, wherein the amount of the at least one RNA transcript decreases by no more than about 5% after the composition is incubated for at least one day at room temperature, preferably wherein the amount of the at least one RNA transcript decreases by no more than about 1%, preferably wherein the amount of the at least one RNA transcript decreases by no more than about 0.5%.

The present disclosure provides kits comprising any one of the preceding stabilization buffers. In some aspects, the stabilization buffer can be in at least one sample collection tube. In some aspects, an at last one sample collection tube can be pre-coated with K2-EDTA. In some aspects, an at least one sample collection tube can be a 6 ml, 16×100 mm sample collection tube. In some aspects, a 6 ml 16×100 mm sample collection tube can be pre-coated with at least about 10.8 mg of K2-EDTA.

The present disclosure provides methods of stabilizing a biological sample from a subject, the method comprising contacting the biological sample and any one of the preceding stabilization buffers, thereby producing a stabilized biological sample. In some aspects, the biological sample can comprise at least one RNA transcript, wherein the expression level of the at least one RNA transcript in the stabilized biological sample measured after incubating the stabilized biological sample for at least 24 hours at about room temperature is within about 5% (±5%), preferably within about 1% (±1%), preferably within about 0.5% (±0.5%) of the expression level of the at least one RNA transcript as measured no more than 1 hour after the sample was collected. In some aspects, the expression level of the at least one RNA transcript can be measured using quantitative PCR.

Methods of the present disclosure can further comprise: i) extracting RNA from the stabilized biological sample; ii) determining the expression level of at least one RNA transcript in the extracted RNA; and iii) diagnosing the subject with a disease and/or disorder, providing a treatment recommendation to the subject, monitoring the progression of a disease and/or disorder in the subject, or administering at least one therapeutic to the subject based on the expression level of the at least one RNA transcript. In some aspects, determining the expression level of an at least one RNA transcript in extracted RNA can comprise the use of quantitative PCR. In some aspects, prior to the use of quantitative PCR, the at least one RNA transcript can be reverse transcribed to produce cDNA.

A disease or disorder can be cancer, gastroenteropancreatic (GEP) neuroendocrine neoplasm (GEP-NEN), melanoma, multiple myeloma, a plasma cell dyscrasia, monoclonal gammopathy of undetermined significance (MGUS), colon cancer, prostate cancer or SARS-CoV-2 infection. A cancer can be a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a gastric cancer, a thyroid cancer, a pancreatic cancer, an esophageal cancer, a prostate cancer, a cervical cancer or a colorectal cancer.

A biological sample can comprise blood, plasma, serum, urine, breast milk, cerebrospinal fluid, mucus, gastric juice, peritoneal fluid, pleural fluid, saliva, sebum, semen, sweat, tears, vaginal secretion, vomit, endolymph, perilymph or any combination thereof. A biological sample can be a blood sample. A blood sample can be a whole blood sample obtained by venipuncture. A blood sample can be a blood sample obtained by finger prick. A biological sample can be a saliva sample.

Any of the above aspects can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a series of graphs showing RNA quality in standard blood that was frozen or in blood samples that were stabilized using compositions and methods of the present disclosure and then incubated for 24-72 hrs at room temperature. The right panel is a graph showing RNA concentration and the right panel is a graph showing RNA integrity number (RIN).

FIG. 2 is a series of graphs showing the number of genes amplified (left panel) and the NETest scores calculated (right panel) from standard blood samples that were frozen or stabilized using compositions and methods of the present disclosure and then incubated for 24-72 hours at room temperature.

FIG. 3 is a series of graphs showing the RNA quality in blood samples of various volumes stabilized using the compositions and methods of the present disclosure. The top left panel shows RNA concentration, the top right panel shows RNA integrity number (RIN), the bottom left panel shows the measured expression of housekeeping genes and the bottom right panel shows in the NETest score calculated in the various samples.

FIG. 4 is a series of graphs showing RNA quality in whole blood (WB) samples or fingerprick blood samples stabilized using the compositions and methods of the present disclosure. The left panel shows the RNA concentration and the right panel shows the RNA integrity number (RIN).

FIG. 5 is a series of graphs showing the correlation between gene expression in whole blood samples and either 50 μl fingerprick blood samples or 35 μl fingerprick blood samples in 6 separate NET patients. The blood samples were stabilized using the methods and compositions of the present disclosure.

FIG. 6 is a graph showing the final NETest scores in whole blood samples or fingerprick blood samples stabilized using the methods and compositions of the present disclosure.

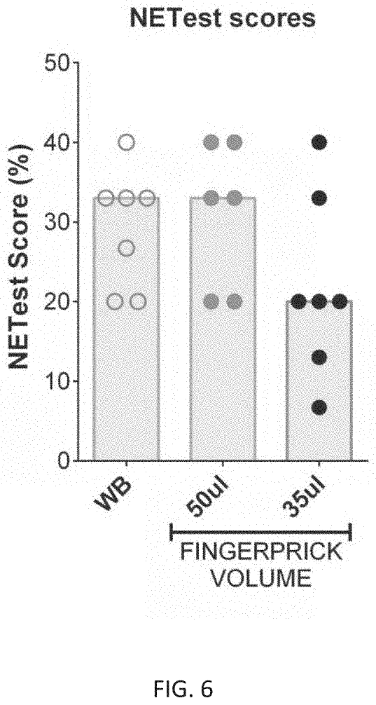

FIG. 7 is a series of graphs showing the correlation between gene expression in blood samples from two separate NET patients after 24 hrs of stabilization at room temperature compared to 48-120 hrs at room temperature (left panel) and the NETest scores calculated from stabilized blood samples as a function of time in 13 neuroendocrine tumor patients and in 6 controls.

FIG. 8 is a graph showing the results of quantitative PCR analysis of saliva samples stabilized using the compositions and methods of the present disclosure over a 120-hour incubation at room temperature.

FIG. 9 is a graph showing the results of quantitative PCR analysis of saliva samples stabilized using the compositions and methods of the present disclosure over a 56-hour thermal excursion with temperatures ranging from 22 to 40° C.

FIG. 10 is a graph showing the results of quantitative PCR analysis of saliva samples stabilized using the compositions and methods of the present disclosure over a 56-hour thermal excursion with temperatures ranging from −10 to 18° C.

### DETAILED DESCRIPTION

Various compositions, kits and methods of the present disclosure are described in full detail herein.

Compositions

The present disclosure provides a buffer comprising at least one chaotropic agent, at least one chelating agent and at least one non-ionic surfactant, wherein the buffer stabilizes a biological sample at about room temperature for at least about one day. As used herein, this buffer can be referred to as a “stabilization buffer”.

In some aspects, an at least one chaotropic agent can be guanidine hydrochloride. In some aspects the guanidine hydrochloride can be present at a concentration of at least about 4.5 M. In some aspects the, guanidine hydrochloride can be present at a concentration of at least about 0.5 M, or at least about 1.0 M, or at least about 1.5 M, or at least about 2.0 M, or at least about 2.5 M, or at least about 3.0 M, or at least about 3.5 M, or at least about 4.0 M, or at least about 4.5 M, or at least about 5.0 M, or at least about 5.5 M, or at least about 6.0 M, or at least about 6.5 M.

In some aspects the guanidine hydrochloride can be present at a concentration of at least 4.5 M. In some aspects the, guanidine hydrochloride can be present at a concentration of at least 0.5 M, or at least 1.0 M, or at least 1.5 M, or at least 2.0 M, or at least 2.5 M, or at least 3.0 M, or at least 3.5 M, or at least 4.0 M, or at least 4.5 M, or at least 5.0 M, or at least 5.5 M, or at least 6.0 M, or at least 6.5 M.

In some aspects, the guanidine hydrochloride can be present at a concentration of about 4.5 M. In some aspects, the guanidine hydrochloride can be present at a concentration of 4.5 M.

In some aspects, the guanidine hydrochloride can be present at a concentration of about 3.6 M to about 5.4 M. In some aspects, the guanidine hydrochloride can be present at a concentration of about 4.05 M to about 4.95 M. In some aspects, the guanidine hydrochloride can be present at a concentration of about 4.275 M to about 4.725 M.

In some aspects, the guanidine hydrochloride can be present at a concentration of 3.6 M to 5.4 M. In some aspects, the guanidine hydrochloride can be present at a concentration of 4.05 M to 4.95 M. In some aspects, the guanidine hydrochloride can be present at a concentration of 4.275 M to 4.725 M.

In some aspects, an at least one non-ionic surfactant can be Triton X-100. In some aspects, the Triton X-100 can be present at a concentration of at least about 0.1% (volume/volume; v/v). In some aspects, the Triton X-100 can be present at a concentration of at least about 0.1% (v/v), or at least about 0.2% (v/v), or at least about 0.3% (v/v), or at least about 0.4% (v/v), or at least about 0.5% (v/v), or at least about 0.6% (v/v), or at least about 0.7% (v/v), or at least about 0.8% (v/v), or at least about 0.9% (v/v), or at least about 1.0% (v/v), or at least about 1.5% (v/v), or at least about 2.0% (v/v), or at least about 2.5% (v/v), or at least about 3.0% (v/v), or at least about 3.5% (v/v), or at least about 4.0% (v/v), or at least about 4.5% (v/v), or at least about 5.0% (v/v), or at least about 5.5% (v/v), or at least about 6.0% (v/v), or at least about 6.5% (v/v), or at least about 7.0% (v/v), or at least about 7.5% (v/v), or at least about 8.0% (v/v), or at least about 8.5% (v/v), or at least about 9.0% (v/v), or at least about 9.5% (v/v), or at least about 10% (v/v).

In some aspects, the Triton X-100 can be present at a concentration of at least 0.1% (v/v). In some aspects, the Triton X-100 can be present at a concentration of at least 0.1% (v/v), or at least 0.2% (v/v), or at least 0.3% (v/v), or at least 0.4% (v/v), or at least 0.5% (v/v), or at least 0.6% (v/v), or at least 0.7% (v/v), or at least 0.8% (v/v), or at least 0.9% (v/v), or at least 1.0% (v/v), or at least 1.5% (v/v), or at least 2.0% (v/v), or at least 2.5% (v/v), or at least 3.0% (v/v), or at least 3.5% (v/v), or at least 4.0% (v/v), or at least 4.5% (v/v), or at least 5.0% (v/v), or at least 5.5% (v/v), or at least 6.0% (v/v), or at least 6.5% (v/v), or at least 7.0% (v/v), or at least 7.5% (v/v), or at least 8.0% (v/v), or at least 8.5% (v/v), or at least 9.0% (v/v), or at least 9.5% (v/v), or at least 10% (v/v).

In some aspects, the Triton X-100 can be present at a concentration of about 0.1% (v/v). In some aspects, the Triton X-100 can be present at a concentration of 0.1% (v/v).

In some aspects, the Triton X-100 can be present at a concentration of about 0.08% to about 0.12% (v/v). In some aspects, the Triton X-100 can be present at a concentration of about 0.09% to about 0.11% (v/v). In some aspects, the Triton X-100 can be present at a concentration of about 0.095% to about 0.105% (v/v).

In some aspects, the Triton X-100 can be present at a concentration of 0.08% to 0.12% (v/v). In some aspects, the Triton X-100 can be present at a concentration of 0.09% to 0.11% (v/v). In some aspects, the Triton X-100 can be present at a concentration of 0.095% to 0.105% (v/v).

In some aspects, an at least one chelating agent can be ethylenediaminetetraacetic acid (EDTA). In some aspects, the EDTA can be present at a concentration of at least about 20 mM. in some aspects, the EDTA can be present at a concentration of at least about 10 mM, or at least about 15 mM, or at least about 20 mM, or at least about 25 mM, or at least about 30 mM, or at least about 35 mM, or at least about 40 mM, or at least about 45 mM, or at least about 50 mM, or at least about 55 mkt, or at least about 60 mM, or at least about 65 mM, or at least about 70 mM, or at least about 75 mM, or at least about 80 mM, or at least about 85 mM, or at least about 90 mM, or at least about 95 mM, or at least about 100 mM.

In some aspects, the EDTA can be present at a concentration of at least 20 mM. in some aspects, the EDTA can be present at a concentration of at least 10 mM, or at least 15 mM, or at least 20 mM, or at least 2.5 mM, or at least 30 mM, or at least 35 mM, or at least 40 mM, or at least 45 mM, or at least 50 mM, or at least 55 mM, or at least 60 mM, or at least 65 mM, or at least 70 mM, or at least 75 mM, or at least 80 mM, or at least 85 mM, or at least 90 mM, or at least 95 mM, or at least 100 mM.

In some aspects, the EDTA can be present at a concentration of about 20 mM, In some aspects, the EDTA can be present at a concentration of 20 mM.

In some aspects, the EDTA can be present at a concentration of about 16 mM to about 24 mM. In some aspects, the EDTA can be present at a concentration of about 18 mM to about 22 mM. In some aspects, the EDTA can be present at a concentration of about 19 mM to about 21 mM.

In some aspects, the EDTA can be present at a concentration of 16 mM to 24 mM. In some aspects, the EDTA can be present at a concentration of 18 mM to 22 mM. In some aspects, the EDTA can be present at a concentration of 19 mM to 21 mM.

In some aspects, a buffer of the present disclosure can be acidic. In some aspects, a buffer of the present disclosure can have an acidic pH. In some aspects, the pH of a buffer can be less than about 4.5. In some aspects, the pH of a buffer can be between about 4.05 and about 4.11. In some aspects, the pH of a buffer can be between about 3.2 and about 4.9. In some aspects, the pH of a buffer can be between about 3.7 and about 4.5. In some aspects, the pH of a buffer can be between about 3.9 and about 4.3. In some aspects, the pH of a buffer can be between 4.05 and 4.11. In some aspects, the pH of a buffer can be between 3.2 and 4.9. In some aspects, the pH of a buffer can be between 3.7 and 4.5. In some aspects, the pH of a buffer can be between 3.9 and 4.3.

In some aspects, the pH of a buffer can be about 4.08. In some aspects, the pH of a buffer can be 4.08.

In some aspects, a buffer of the present disclosure, a buffer can comprise sodium citrate. The sodium citrate can be present at a concentration of at least about 120 mM. In some aspects, the sodium citrate can be present at a concentration of at least about 80 mM, or at least about 90 mM, or at least about 100 mM, or at least about 110 mM, or at least about 120 mM, or at least about 130 mM, or at least about 140 mM, or at least about 150 mM, or at least about 160 mM.

The sodium citrate can be present at a concentration of at least 120 mM. In some aspects, the sodium citrate can be present at a concentration of at least 80 mM, or at least 90 mM, or at least 100 mM, or at least 110 mM, or at least 120 mM, or at least 130 mM, or at least 140 mM, or at least 150 mM, or at least 160 mM.

The sodium citrate can be present at a concentration of about 120 mM, The sodium citrate can be present at a concentration of 120 mM.

In some aspects, the sodium citrate can be present at a concentration of about 96 mM to about 144 mM. In some aspects, the sodium citrate can be present at a concentration of about 108 mM to about 132 mM, In some aspects, the sodium citrate can be present at a concentration of about 114 mM to about 126 mM.

In some aspects, the sodium citrate can be present at a concentration of 96 mM to 144 mM. In some aspects, the sodium citrate can be present at a concentration of 108 mM to 132 mM. In some aspects, the sodium citrate can be present at a concentration of 114 mM to 126 mM.

In some aspects, a buffer of the present disclosure, a buffer can comprise citric acid. The citric acid can be present in a concentration of at least about 80 mM. In some aspects, the citric acid can be present at a concentration of at least about 40 mM, or at least about 50 mM, or at least about 60 mM, or at least about 70 mM, or at least about 80 mM, or at least about 90 mM, or at least about 100 mM, or at least about 110 mM, or at least about 120 mM.

In some aspects, the citric acid can be present at a concentration of at least 40 mM, or at least 50 mM, or at least 60 mM, or at least 70 mM, or at least 80 mM, or at least 90 mM, or at least 100 mM, or at least 110 mM, or at least 120 mM.

The citric acid can be present in a concentration of about 80 mM. The citric acid can be present in a concentration of 80 mM.

In some aspects, the citric acid can be present at a concentration of about 64 mM to about 96 mM. In some aspects, the citric acid can be present at a concentration of about 72 mM to about 88 mM. In some aspects, the citric acid can be present at a concentration of about 76 mM to about 84 mM.

In some aspects, the citric acid can be present at a concentration of 64 mM to 96 mM, some aspects, the citric acid can be present at a concentration of 72 mM to 88 mM. In some aspects, the citric acid can be present at a concentration of 76 mM to 84 mM.

In some aspects, the present disclosure provides a stabilization buffer comprising: (a) guanidine hydrochloride at a concentration of at least about 4.5 M; (b) Triton X-100 at a concentration of at least about 0.1% (volume/volume); and (c) EDTA at a concentration of at least about 20 mM, wherein the pH of the stabilization buffer is less than about 4.5.

In some aspects, the present disclosure provides a stabilization buffer comprising: (a) guanidine hydrochloride at a concentration of about 4.5 M; (b) Triton X-100 at a concentration of about 0.1% (volume/volume); and (c) EDTA at a concentration of about 20 mM, wherein the pH of the stabilization buffer is less than about 4.5.

In some aspects, the present disclosure provides a stabilization buffer comprising: (a) guanidine hydrochloride at a concentration of 4.5 M; (b) Triton X-100 at a concentration of 0.1% (volume/volume); and (c) EDTA at a concentration of 20 mM, wherein the pH of the stabilization buffer is less than 4.5.

In some aspects, the present disclosure provides a stabilization buffer comprising: (a) guanidine hydrochloride at a concentration of at least about 4.5 M; (b) Triton X-100 at a concentration of at least about 0.1% (volume/volume); and (c) EDTA at a concentration of at least about 20 mM, wherein the pH of the stabilization buffer is between about 4.05 and about 4.11.

In some aspects, the present disclosure provides a stabilization buffer comprising: (a) guanidine hydrochloride at a concentration of about 4.5 M; (b) Triton X-100 at a concentration of about 0.1% (volume/volume); and (c) EDTA at a concentration of about 20 mM, wherein the pH of the stabilization buffer is between about 4.05 and about 4.11.

In some aspects, the present disclosure provides a stabilization buffer comprising: (a) guanidine hydrochloride at a concentration of 4.5 M; (b) Triton X-100 at a concentration of 0.1% (volume/volume); and (c) EDTA at a concentration of 20 mM, wherein the pH of the stabilization buffer is between 4.05 and 4.11.

In some aspects, the present disclosure provides a stabilization buffer comprising (a) guanidine hydrochloride at a concentration of about 3.6 M to about 5.4 M; (b) Triton X-100 at a concentration of about 0.08% to about 0.12% (v/v); and (c) EDTA at a concentration of about 16 mM to about 24 mM, wherein the pH of the stabilization buffer is less than about 4.5. In some aspects, the present disclosure provides a stabilization buffer comprising (a) guanidine hydrochloride at a concentration of about 4.05 M to about 4.95 M; (b) Triton X-100 at a concentration of about 0.09% to about 0.11% (v/v); and (c) EDTA at a concentration of about 18 mM to about 22 mM, wherein the pH of the stabilization buffer is less than about 4.5, In some aspects, the present disclosure provides a stabilization buffer comprising (a) guanidine hydrochloride at a concentration of about 4.275 M to about 4.725 M; (b) Triton X-100 at a concentration of about 0.095% to about 0.105% (v/v); and (c) EDTA at a concentration of about 19 mM to about 21 mM, wherein the pH of the stabilization buffer is less than about 4.5.

In some aspects, the present disclosure provides a stabilization buffer comprising (a) guanidine hydrochloride at a concentration of about 3.6 M to about 5.4 M; (b) Triton X-100 at a concentration of about 0.08% to about 0.12% (v/v); and (c) EDTA at a concentration of about 16 mM to about 24 mM, wherein the pH of the stabilization buffer is between about 4.05 and about 4.11. In some aspects, the present disclosure provides a stabilization buffer comprising (a) guanidine hydrochloride at a concentration of about 4.05 M to about 4.95 M; (b) Triton X-100 at a concentration of about 0.09% to about 0.11% (v/v); and (c) EDTA at a concentration of about 18 mM to about 22 mM, wherein the pH of the stabilization buffer is between about 4.05 and about 4.11. In some aspects, the present disclosure provides a stabilization buffer comprising (a) guanidine hydrochloride at a concentration of about 4.275 M to about 4.725 M; (b) Triton X-100 at a concentration of about 0.095% to about 0.105% (v/v); and (c) EDTA at a concentration of about 19 mM to about 21 mM, wherein the pH of the stabilization buffer is between about 4.05 and about 4.11.

In some aspects, the present disclosure provides a stabilization buffer comprising (a) guanidine hydrochloride at a concentration of 3.6 M to 5.4 M; (b) Triton X-100 at a concentration of 0.08% to 0.12% (v/v); and (c) EDTA at a concentration of 16 mM to 24 mM, wherein the pH of the stabilization buffer is less than 4.5. In some aspects, the present disclosure provides a stabilization buffer comprising (a) guanidine hydrochloride at a concentration of 4.05 M to 4.95 M; (b) Triton X-100 at a concentration of 0.09% to 0.11% (v/v); and (c) EDTA at a concentration of 18 mM to 22 mM, wherein the pH of the stabilization buffer is less than 4.5. In some aspects, the present disclosure provides a stabilization buffer comprising (a) guanidine hydrochloride at a concentration of 4.275 M to 4.725 M; (b) Triton X-100 at a concentration of 0.095% to 0.105% (v/v); and (c) EDTA at a concentration of 19 mM to 21 mM, wherein the pH of the stabilization buffer is less than 4.5.

In some aspects, the present disclosure provides a stabilization buffer comprising (a) guanidine hydrochloride at a concentration of 3.6 M to 5.4 M; (b) Triton X-100 at a concentration of 0.08% to 0.12% (v/v); and (c) EDTA at a concentration of 16 mM to 24 mM, wherein the pH of the stabilization buffer is between 4.05 and 4.11. In some aspects, the present disclosure provides a stabilization buffer comprising (a) guanidine hydrochloride at a concentration of 4.05 M to 4.95 M; (b) Triton X-100 at a concentration of 0.09% to 0.11% (v/v); and (c) EDTA at a concentration of 18 mM to 22 mM, wherein the pH of the stabilization buffer is between 4.05 and 4.11. In some aspects, the present disclosure provides a stabilization buffer comprising (a) guanidine hydrochloride at a concentration of 4.275 M to 4.725 M; (b) Triton X-100 at a concentration of 0.095% to 0.105% (v/v); and (c) EDTA at a concentration of 19 mM to 21 mM, wherein the pH of the stabilization buffer is between 4.05 and 4.11.

In some aspects, the present disclosure provides a stabilization buffer comprising: (a) guanidine hydrochloride at a concentration of at least about 4.5 M; (b) Triton X-100 at a concentration of at least about 0.1% (volume/volume); (c) EDTA at a concentration of at least about 20 mM; (d) citric acid at a concentration of at least about 80 mM; and (e) sodium citrate at a concentration of at least about 120 mM, wherein the pH of the stabilization buffer is less than about 4.5.

In some aspects, the present disclosure provides a stabilization buffer comprising: (a) guanidine hydrochloride at a concentration of about 4.5 M; (b) Triton X-100 at a concentration of about 0.1% (volume/volume); (c) EDTA at a concentration of about 20 mM; (d) citric acid at a concentration of about 80 mM; and (e) sodium citrate at a concentration of about 120 mM, wherein the pH of the stabilization buffer is less than about 4.5.

In some aspects, the present disclosure provides a stabilization buffer comprising: (a) guanidine hydrochloride at a concentration of 4.5 M; (b) Triton X-100 at a concentration of 0.1% (volume/volume); (c) EDTA at a concentration of 2.0 mM; (d) citric acid at a concentration of 80 mM; and (e) sodium citrate at a concentration of 120 mM, wherein the pH of the stabilization buffer is less than 4.5.

In some aspects, the present disclosure provides a stabilization buffer comprising: (a) guanidine hydrochloride at a concentration of at least about 4.5 M; (b) Triton X-100 at a concentration of at least about 0.1% (volume/volume); (c) EDTA at a concentration of at least about 20 mM; (d) citric acid at a concentration of at least about 80 mM; and (e) sodium citrate at a concentration of at least about 120 mM, wherein the pH of the buffer between about 4.05 and about 4.11.

In some aspects, the present disclosure provides a stabilization buffer comprising: (a) guanidine hydrochloride at a concentration of about 4.5 M; (b) Triton X-100 at a concentration of about 0.1% (volume/volume); (c) EDTA at a concentration of about 20 mM; (d) citric acid at a concentration of about 80 mM; and (e) sodium citrate at a concentration of about 120 mM, wherein the pH of the buffer between about 4.05 and about 4.11.

In some aspects, the present disclosure provides a stabilization buffer comprising: (a) guanidine hydrochloride at a concentration of 4.5 M; (b) Triton X-100 at a concentration of 0.1% (volume/volume); (c) EDTA at a concentration of 20 mM; (d) citric acid at a concentration of 80 mM; and (e) sodium citrate at a concentration of 120 mM, wherein the pH of the buffer between 4.05 and 4.11.

In some aspects, the present disclosure provides a stabilization buffer comprising (a) guanidine hydrochloride at a concentration of about 3.6 M to about 5.4 M; (b) Triton X-100 at a concentration of about 0.08% to about 0.12% (v/v); (c) EDTA at a concentration of about 16 mM to about 24 mM; (d) citric acid at a concentration of about 64 mM to about 96 mM; and (e) sodium citrate at a concentration of about 96 mM to about 144 mM, wherein the pH of the stabilization buffer is less than about 4.5. In some aspects, the present disclosure provides a stabilization buffer comprising (a) guanidine hydrochloride at a concentration of about 4.05 M to about 4.95 M; (b) Triton X-100 at a concentration of about 0.09% to about 0.11% (v/v); (c) EDTA at a concentration of about 18 mM to about 22 mM; (d) citric acid at a concentration of about 72 mM to about 88 mM; and (e) sodium citrate at a concentration of about 108 mM to about 132 mM, wherein the pH of the stabilization buffer is less than about 4.5. In some aspects, the present disclosure provides a stabilization buffer comprising (a) guanidine hydrochloride at a concentration of about 4.275 M to about 4.725 M; (b) Triton X-100 at a concentration of about 0.095% to about 0.105% (v/v); (c) EDTA at a concentration of about 19 mM to about 21 mM; and (d) citric acid at a concentration of about 76 mM to about 84 mM; and (e) sodium citrate at a concentration of about 114 mM to about 126 mM, wherein the pH of the stabilization buffer is less than about 4.5.

In some aspects, the present disclosure provides a stabilization buffer comprising (a) guanidine hydrochloride at a concentration of about 3.6 M to about 5.4 M; (b) Triton X-100 at a concentration of about 0.08% to about 0.12% (v/v); (c) EDTA at a concentration of about 16 mM to about 24 mM; (d) citric add at a concentration of about 64 mM to about 96 mM; and (e) sodium citrate at a concentration of about 96 mM to about 144 mM, wherein the pH of the stabilization buffer is between about 4.05 and about 4.11. In some aspects, the present disclosure provides a stabilization buffer comprising (a) guanidine hydrochloride at a concentration of about 4.05 M to about 4.95 M; (b) Triton X-100 at a concentration of about 0.09% to about 0.11% (v/v); (c) EDTA at a concentration of about 18 mM to about 22 mM; (d) citric acid at a concentration of about 72 mM to about 88 mM; and (e) sodium citrate at a concentration of about 108 mM to about 132 mM, wherein the pH of the stabilization buffer is between about 4.05 and about 4.11. In some aspects, the present disclosure provides a stabilization buffer comprising (a) guanidine hydrochloride at a concentration of about 4.275 M to about 4.725 M; (b) Triton X-100 at a concentration of about 0.095% to about 0.105% (v/v); (c) EDTA at a concentration of about 19 mM to about 21 mM; and (d) citric acid at a concentration of about 76 mM to about 84 mM; and (e) sodium citrate at a concentration of about 114 mM to about 126 mM, wherein the pH of the stabilization buffer is between about 4.05 and about 4.11.

In some aspects, the present disclosure provides a stabilization buffer comprising (a) guanidine hydrochloride at a concentration of 3.6 M to 5.4 M; (b) Triton X-100 at a concentration of 0.08% to 0.12% (v/v); (c) EDTA at a concentration of 16 mM to 24 mM; (d) citric acid at a concentration of 64 mM to 96 mM; and (e) sodium citrate at a concentration of 96 mM to 144 mM, wherein the pH of the stabilization buffer is less than 4.5. In some aspects, the present disclosure provides a stabilization buffer comprising (a) guanidine hydrochloride at a concentration of 4.05 M to 4.95 M; (b) Triton X-100 at a concentration of 0.09% to 0.11% (v/v); (c) EDTA at a concentration of 18 mM to 22 mM; (d) citric acid at a concentration of 72 mM to 88 mM; and (e) sodium citrate at a concentration of 108 mM to 132 mM, wherein the pH of the stabilization buffer is less than 4.5. In some aspects, the present disclosure provides a stabilization buffer comprising (a) guanidine hydrochloride at a concentration of 4.275 M to 4.725 M; (b) Triton X-100 at a concentration of 0.095% to 0.105% (v/v); (c) EDTA at a concentration of 19 mM to 21 mM; and (d) citric acid at a concentration of 76 mM to 84 mM; and (e) sodium citrate at a concentration of 114 mM to 126 mM, wherein the pH of the stabilization buffer is less than 4.5.

In some aspects, the present disclosure provides a stabilization buffer comprising (a) guanidine hydrochloride at a concentration of 3.6 M to 5.4 M; (b) Triton X-100 at a concentration of 0.08% to 0.12% (v/v); (c) EDTA at a concentration of 16 mM to 24 mM; (d) citric acid at a concentration of 64 mM to 96 mM; and (e) sodium citrate at a concentration of 96 mM to 144 mM, wherein the pH of the stabilization buffer is between 4.05 and 4.11. In some aspects, the present disclosure provides a stabilization buffer comprising (a) guanidine hydrochloride at a concentration of 4.05 M to 4.95 M; (b) Triton X-100 at a concentration of 0.09% to 0.11% (v/v); (c) EDTA at a concentration of 18 mM to 22 mM; (d) citric acid at a concentration of 72 mM to 88 mM; and (e) sodium citrate at a concentration of 108 mM to 132 mM, wherein the pH of the stabilization buffer is between 4.05 and 4.11. In some aspects, the present disclosure provides a stabilization buffer comprising (a) guanidine hydrochloride at a concentration of 4.275 M to 4.725 M; (b) Triton X-100 at a concentration of 0.095% to 0.105% (v/v); (c) EDTA at a concentration of 19 mM to 21 mM; and (d) citric acid at a concentration of 76 mM to 84 mM; and (e) sodium citrate at a concentration of 114 mM to 126 mM, wherein the of the stabilization buffer is between 4.05 and 4.11.

Methods

The present disclosure provides methods of stabilizing a biological sample from a subject, the methods comprising contacting the biological sample and a stabilization buffer of the present disclosure to produce a stabilized biological sample.

The present disclosure provides methods of stabilizing a biological sample from a subject, the methods comprising contacting the biological sample and a stabilization buffer of the present disclosure to produce a stabilized biological sample, such that the amount of at least one RNA transcript in the stabilized biological sample decreases by no more than about 0.1%, or no more than about 0.5%, or no more than about 1%, or no more than about 2.5%, or no more than about 5%, or no more than about 10% after incubating the stabilized biological sample for a given time period at a given temperature or range of temperatures. In some aspects, the given time period can be about 24 hours, or about 48 hours, or about 72 hours, or at about 4 days, or about 5 days, or about 6 days, or about 7 days, or about 8 days, or about 9 days, or about 10 days, or about 11 days, or about 12 days, or about 13 days, or about 14 days, or about 15 days, or about 16 days, or about 17 days, or about 18 days, or about 19 days, or about 20 days, or about 21 days, or about 22 days, or about 23 days, or about 24 days, or about 25 days, or about 26 days, or about 27 days, or about 28 days, or about 29 days, or about 30 days. In some aspects, the given temperature or range of temperatures can be about room temperature, as defined herein. In some aspects, the given temperature or range of temperatures can be any temperature in the range of −10° C. to 18° C. In some aspects, the given temperature or range of temperatures can be any temperature in the range of −22° C. to 40° C.

Stabilizing a biological sample can comprise contacting the biological sample with a stabilization buffer of the present disclosure such that the stabilization buffer prevents the degradation of RNA within the stabilized biological sample. Stabilizing a biological sample can comprise contacting the biological sample with a stabilization buffer of the present disclosure such that the stabilization buffer prevents the degradation of RNA within the stabilized biological sample such that the degradation of the RNA, over a given time period at a given temperature or range of temperatures, is decreased by at least about 10%, or at least about 20%, or at least about 30%, or at least about 40%, or at least about 50%, or at least about 60%, or at least about 70%, or at least about 80%, or at least about 90%, or at least about 95%, or at least about 99.5% or at least about 100% as compared to RNA degradation in a corresponding biological sample that was not contacted with the stabilization buffer over the same time period at the same temperature. In some aspects, the given time period can be about 24 hours, or about 48 hours, or about 72 hours, or at about 4 days, or about 5 days, or about 6 days, or about 7 days, or about 8 days, or about 9 days, or about 10 days, or about 11 days, or about 12 days, or about 13 days, or about 14 days, or about 15 days, or about 16 days, or about 17 days, or about 18 days, or about 19 days, or about 20 days, or about 21 days, or about 22 days, or about 23 days, or about 24 days, or about 25 days, or about 26 days, or about 27 days, or about 28 days, or about 29 days, or about 30 days. In some aspects, the given temperature or range of temperatures can be about room temperature, as defined herein. In some aspects, the given temperature or range of temperatures can be any temperature in the range of −10° C. to 18° C. In some aspects, the given temperature or range of temperatures can be any temperature in the range of −22° C. to 40° C.

Stabilizing a biological sample can comprise contacting the biological sample with a stabilization buffer of the present disclosure such that the stabilization buffer prevents the degradation of RNA within the stabilized biological sample, such that the expression level of at least one RNA transcript in the stabilized biological sample measured after incubating the stabilized biological sample for a given time period at a given temperature or range of temperatures is within about 10% (±10%) of the expression level of the at least one RNA transcript as measured no more than 1 hour after the sample was collected. Stabilizing a biological sample can comprise contacting the biological sample with a stabilization buffer of the present disclosure such that the stabilization buffer prevents the degradation of RNA within the stabilized biological sample, such that the expression level of at least one RNA transcript in the stabilized biological sample measured after incubating the stabilized biological sample for a given time period at a given temperature or range of temperatures is within about 5% (±5%) of the expression level of the at least one RNA transcript as measured no more than 1 hour after the sample was collected. Stabilizing a biological sample can comprise contacting the biological sample with a stabilization buffer of the present disclosure such that the stabilization buffer prevents the degradation of RNA within the stabilized biological sample, such that the expression level of at least one RNA transcript in the stabilized biological sample measured after incubating the stabilized biological sample for a given time period at a given temperature or range of temperatures is within about 2.5% (±2.5%) of the expression level of the at least one RNA transcript as measured no more than 1 hour after the sample was collected. Stabilizing a biological sample can comprise contacting the biological sample with a stabilization buffer of the present disclosure such that the stabilization buffer prevents the degradation of RNA within the stabilized biological sample, such that the expression level of at least one RNA transcript in the biological sample measured after incubating the stabilized biological sample for a given time period at a given temperature or range of temperatures is within about 1% (±1%) of the expression level of the at least one RNA transcript as measured no more than 1 hour after the sample was collected. Stabilizing a biological sample can comprise contacting the biological sample with a stabilization buffer of the present disclosure such that the stabilization buffer prevents the degradation of RNA within the biological sample, such that the expression level of at least one RNA transcript in the stabilized biological sample measured after incubating the stabilized biological sample for a given time period at a given temperature or range of temperatures is within about 0.5% (±0.5%) of the expression level of the at least one RNA transcript as measured no more than 1 hour after the sample was collected. Stabilizing a biological sample can comprise contacting the biological sample with a stabilization buffer of the present disclosure such that the stabilization buffer prevents the degradation of RNA within the biological sample, such that the expression level of at least one RNA transcript in the stabilized biological sample measured after incubating the stabilized biological sample for a given time period at a given temperature or range of temperatures is within about 0.1% (±0.1%) of the expression level of the at least one RNA transcript as measured no more than 1 hour after the sample was collected. In some aspects, the given time period can be about 24 hours, or about 48 hours, or about 72 hours, or at about 4 days, or about 5 days, or about 6 days, or about 7 days, or about 8 days, or about 9 days, or about 10 days, or about 11 days, or about 12 days, or about 13 days, or about 14 days, or about 15 days, or about 16 days, or about 17 days, or about 18 days, or about 19 days, or about 20 days, or about 21 days, or about 22 days, or about 23 days, or about 24 days, or about 25 days, or about 26 days, or about 27 days, or about 28 days, or about 29 days, or about 30 days. In some aspects, the given temperature or range of temperatures can be about room temperature, as defined herein. In some aspects, the given temperature or range of temperatures can be any temperature in the range of −10° C. to 18° C. In some aspects, the given temperature or range of temperatures can be any temperature in the range of −22° C. to 40° C.

Stabilizing a biological sample can comprise contacting the biological sample with a stabilization buffer of the present disclosure such that the stabilization buffer prevents the degradation of RNA within the stabilized biological sample, such that the expression level of at least one RNA transcript in the stabilized biological sample measured after incubating the stabilized biological sample for a given time period at a given temperature or range of temperatures is within about 10% (±10%) of the expression level of the at least one RNA transcript as measured no more than 12 hours after the sample was collected. Stabilizing a biological sample can comprise contacting the biological sample with a stabilization buffer of the present disclosure such that the stabilization buffer prevents the degradation of RNA within the stabilized biological sample, such that the expression level of at least one RNA transcript in the stabilized biological sample measured after incubating the stabilized biological sample for a given time period at a given temperature or range of temperatures is within about 5% (±5%) of the expression level of the at least one RNA transcript as measured no more than 12 hours after the sample was collected. Stabilizing a biological sample can comprise contacting the biological sample with a stabilization buffer of the present disclosure such that the stabilization buffer prevents the degradation of RNA within the stabilized biological sample, such that the expression level of at least one RNA transcript in the stabilized biological sample measured after incubating the stabilized biological sample for a given time period at a given temperature or range of temperatures is within about 2.5% (±2.5%) of the expression level of the at least one RNA transcript as measured no more than 12 hours after the sample was collected. Stabilizing a biological sample can comprise contacting the biological sample with a stabilization buffer of the present disclosure such that the stabilization buffer prevents the degradation of RNA within the stabilized biological sample, such that the expression level of at least one RNA transcript in the biological sample measured after incubating the stabilized biological sample for a given time period at a given temperature or range of temperatures is within about 1% (±1%) of the expression level of the at least one RNA transcript as measured no more than 12 hours after the sample was collected. Stabilizing a biological sample can comprise contacting the biological sample with a stabilization buffer of the present disclosure such that the stabilization buffer prevents the degradation of RNA within the biological sample, such that the expression level of at least one RNA transcript in the stabilized biological sample measured after incubating the stabilized biological sample for a given time period at a given temperature or range of temperatures is within about 0.5% (±0.5%) of the expression level of the at least one RNA transcript as measured no more than 12 hours after the sample was collected. Stabilizing a biological sample can comprise contacting the biological sample with a stabilization buffer of the present disclosure such that the stabilization buffer prevents the degradation of RNA within the biological sample, such that the expression level of at least one RNA transcript in the stabilized biological sample measured after incubating the stabilized biological sample for a given time period at a given temperature or range of temperatures is within about 0.1% (±0.1%) of the expression level of the at least one RNA transcript as measured no more than 12 hours after the sample was collected. In some aspects, the given time period can be about 24 hours, or about 48 hours, or about 72 hours, or at about 4 days, or about 5 days, or about 6 days, or about 7 days, or about 8 days, or about 9 days, or about 10 days, or about 11 days, or about 12 days, or about 13 days, or about 14 days, or about 15 days, or about 16 days, or about 17 days, or about 18 days, or about 19 days, or about 20 days, or about 21 days, or about 22 days, or about 23 days, or about 24 days, or about 25 days, or about 26 days, or about 27 days, or about 28 days, or about 29 days, or about 30 days. In some aspects, the given temperature or range of temperatures can be about room temperature, as defined herein. In some aspects, the given temperature or range of temperatures can be any temperature in the range of −10° C. to 18° C. In some aspects, the given temperature or range of temperatures can be any temperature in the range of −22° C. to 40° C.

In some aspects, the expression level of the at least one RNA transcript is measured using quantitative PCR.

Stabilizing a biological sample can comprise contacting the biological sample with a stabilization buffer of the present disclosure such that the stabilization buffer prevents the degradation of RNA within the stabilized biological sample, such that the RNA integrity number (RIN) of RNA extracted from the stabilized biological sample after incubating the stabilized biological sample for a given time period at a given temperature or range of temperatures is within about 10% (±10%) of the RIN of RNA extracted from the biological sample no more than 1 hour after the sample was collected. Stabilizing a biological sample can comprise contacting the biological sample with a stabilization buffer of the present disclosure such that the stabilization buffer prevents the degradation of RNA within the stabilized biological sample, such that the RNA integrity number (RIN) of RNA extracted from the stabilized biological sample after incubating the stabilized biological sample for a given time period at a given temperature or range of temperatures is within about 5% (±5%) of the RIN of RNA extracted from the biological sample no more than 1 hour after the sample was collected. Stabilizing a biological sample can comprise contacting the biological sample with a stabilization buffer of the present disclosure such that the stabilization buffer prevents the degradation of RNA within the stabilized biological sample, such that the RNA integrity number (RIN) of RNA extracted from the stabilized biological sample after incubating the stabilized biological sample for a given time period at a given temperature or range of temperatures is within about 2.5% (±2.5%) of the RIN of RNA extracted from the biological sample no more than 1 hour after the sample was collected. Stabilizing a biological sample can comprise contacting the biological sample with a stabilization buffer of the present disclosure such that the stabilization buffer prevents the degradation of RNA within the stabilized biological sample, such that the RNA integrity number (RIN) of RNA extracted from the stabilized biological sample after incubating the stabilized biological sample for a given time period at a given temperature or range of temperatures is within about 1% (±1%) of the RIN of RNA extracted from the biological sample no more than 1 hour after the sample was collected. Stabilizing a biological sample can comprise contacting the biological sample with a stabilization buffer of the present disclosure such that the stabilization buffer prevents the degradation of RNA within the stabilized biological sample, such that the RNA integrity number (RIN) of RNA extracted from the stabilized biological sample after incubating the stabilized biological sample for a given time period at a given temperature or range of temperatures is within about 0.5% (±0.5%) of the RIN of RNA extracted from the biological sample no more than 1 hour after the sample was collected. Stabilizing a biological sample can comprise contacting the biological sample with a stabilization buffer of the present disclosure such that the stabilization buffer prevents the degradation of RNA within the stabilized biological sample, such that the RNA integrity number (RIN) of RNA extracted from the stabilized biological sample after incubating the stabilized biological sample for a given time period at a given temperature or range of temperatures is within about 0.1% (±0.1%) of the RIN of RNA extracted from the biological sample no more than 1 hour after the sample was collected. In some aspects, the given time period can be about 24 hours, or about 48 hours, or about 72 hours, or at about 4 days, or about 5 days, or about 6 days, or about 7 days, or about 8 days, or about 9 days, or about 10 days, or about 11 days, or about 12 days, or about 13 days, or about 14 days, or about 15 days, or about 16 days, or about 17 days, or about 18 days, or about 19 days, or about 20 days, or about 21 days, or about 22 days, or about 23 days, or about 24 days, or about 25 days, or about 26 days, or about 27 days, or about 28 days, or about 29 days, or about 30 days. In some aspects, the given temperature or range of temperatures can be about room temperature, as defined herein. In some aspects, the given temperature or range of temperatures can be any temperature in the range of −10° C. to 18° C. In some aspects, the given temperature or range of temperatures can be any temperature in the range of −22° C. to 40° C.

Stabilizing a biological sample can comprise contacting the biological sample with a stabilization buffer of the present disclosure such that the stabilization buffer prevents the degradation of RNA within the stabilized biological sample, such that the RNA integrity number (RIN) of RNA extracted from the stabilized biological sample after incubating the stabilized biological sample for a given time period at a given temperature or range of temperatures is within about 10% (±10%) of the RIN of RNA extracted from the biological sample no more than 12 hours after the sample was collected. Stabilizing a biological sample can comprise contacting the biological sample with a stabilization buffer of the present disclosure such that the stabilization buffer prevents the degradation of RNA within the stabilized biological sample, such that the RNA integrity number (RIN) of RNA extracted from the stabilized biological sample after incubating the stabilized biological sample for a given time period at a given temperature or range of temperatures is within about 5% (±5%) of the RIN of RNA extracted from the biological sample no more than 12 hours after the sample was collected. Stabilizing a biological sample can comprise contacting the biological sample with a stabilization buffer of the present disclosure such that the stabilization buffer prevents the degradation of RNA within the stabilized biological sample, such that the RNA integrity number (RIN) of RNA extracted from the stabilized biological sample after incubating the stabilized biological sample for a given time period at a given temperature or range of temperatures is within about 2.5% (±2.5%) of the RIN of RNA extracted from the biological sample no more than 12 hours after the sample was collected. Stabilizing a biological sample can comprise contacting the biological sample with a stabilization buffer of the present disclosure such that the stabilization buffer prevents the degradation of RNA within the stabilized biological sample, such that the RNA integrity number (RIN) of RNA extracted from the stabilized biological sample after incubating the stabilized biological sample for a given time period at a given temperature or range of temperatures is within about 1% (±1%) of the RIN of RNA extracted from the biological sample no more than 12 hours after the sample was collected. Stabilizing a biological sample can comprise contacting the biological sample with a stabilization buffer of the present disclosure such that the stabilization buffer prevents the degradation of RNA within the stabilized biological sample, such that the RNA integrity number (RIN) of RNA extracted from the stabilized biological sample after incubating the stabilized biological sample for a given time period at a given temperature or range of temperatures is within about 0.5% (±0.5%) of the RIN of RNA extracted from the biological sample no more than 12 hours after the sample was collected. Stabilizing a biological sample can comprise contacting the biological sample with a stabilization buffer of the present disclosure such that the stabilization buffer prevents the degradation of RNA within the stabilized biological sample, such that the RNA integrity number (RIN) of RNA extracted from the stabilized biological sample after incubating the stabilized biological sample for a given time period at a given temperature or range of temperatures is within about 0.1% (±0.1%) of the RIN of RNA extracted from the biological sample no more than 12 hours after the sample was collected. In some aspects, the given time period can be about 24 hours, or about 48 hours, or about 72 hours, or at about 4 days, or about 5 days, or about 6 days, or about 7 days, or about 8 days, or about 9 days, or about 10 days, or about 11 days, or about 12 days, or about 13 days, or about 14 days, or about 15 days, or about 16 days, or about 17 days, or about 18 days, or about 19 days, or about 20 days, or about 21 days, or about 22 days, or about 23 days, or about 24 days, or about 25 days, or about 26 days, or about 27 days, or about 28 days, or about 29 days, or about 30 days. In some aspects, the given temperature or range of temperatures can be about room temperature, as defined herein. In some aspects, the given temperature or range of temperatures can be any temperature in the range of −10° C. to 18° C. In some aspects, the given temperature or range of temperatures can be any temperature in the range of −22° C. to 40° C.

RIN values can be calculated using methods and techniques standard in the art, as would be appreciated by the skilled artisan

In some aspects, the biological sample can be a blood sample. In some aspects, a blood sample can be a venipuncture blood sample. In some aspects, a blood sample can be a finger prick blood sample. In some aspects, the biological sample can be a saliva sample. In some aspects, the sample can be a nasopharyngeal swab sample. In some aspects, the biological sample can be a bodily fluid sample. A bodily fluid sample can comprise blood, plasma, serum, urine, breast milk, cerebrospinal fluid, mucus, gastric juice, peritoneal fluid, pleural fluid, saliva, sebum, semen, sweat, tears, vaginal secretion, vomit, endolymph, perilymph or any combination thereof.

The present disclosure provides methods of stabilizing a blood sample from a subject, the methods comprising contacting the blood sample and a stabilization buffer of the present disclosure to produce a stabilized blood sample.

The present disclosure provides methods of stabilizing a blood sample from a subject, the methods comprising contacting the blood sample and a stabilization buffer of the present disclosure to produce a stabilized blood sample, such that the amount of at least one RNA transcript in the stabilized blood sample decreases by no more than about 0.1%, or no more than about 0.5%, or no more than about 1%, or no more than about 2.5%, or no more than about 5%, or no more than about 10% after incubating the stabilized blood sample for a given time period at a given temperature or range of temperatures. In some aspects, the given time period can be about 24 hours, or about 48 hours, or about 72 hours, or at about 4 days, or about 5 days, or about 6 days, or about 7 days, or about 8 days, or about 9 days, or about 10 days, or about 11 days, or about 12 days, or about 13 days, or about 14 days, or about 15 days, or about 16 days, or about 17 days, or about 18 days, or about 19 days, or about 20 days, or about 21 days, or about 22 days, or about 23 days, or about 24 days, or about 25 days, or about 26 days, or about 27 days, or about 28 days, or about 29 days, or about 30 days. In some aspects, the given temperature or range of temperatures can be about room temperature, as defined herein. In some aspects, the given temperature or range of temperatures can be any temperature in the range of −10° C. to 18° C. In some aspects, the given temperature or range of temperatures can be any temperature in the range of −22° C. to 40° C.

The present disclosure provides methods of stabilizing a saliva sample from a subject, the methods comprising contacting the saliva sample and a stabilization buffer of the present disclosure to produce a stabilized saliva sample, such that the amount of at least one RNA transcript in the stabilized saliva sample decreases by no more than about 0.1%, or no more than about 0.5%, or no more than about 1%, or no more than about 2.5%, or no more than about 5%, or no more than about 10% after incubating the stabilized saliva sample for a given time period at a given temperature or range of temperatures. In some aspects, the given time period can be about 24 hours, or about 48 hours, or about 72 hours, or at about 4 days, or about 5 days, or about 6 days, or about 7 days, or about 8 days, or about 9 days, or about 10 days, or about 11 days, or about 12 days, or about 13 days, or about 14 days, or about 15 days, or about 16 days, or about 17 days, or about 18 days, or about 19 days, or about 20 days, or about 21 days, or about 22 days, or about 23 days, or about 24 days, or about 25 days, or about 26 days, or about 27 days, or about 28 days, or about 29 days, or about 30 days. In some aspects, the given temperature or range of temperatures can be about room temperature, as defined herein. In some aspects, the given temperature or range of temperatures can be any temperature in the range of −10° C. to 18° C. In some aspects, the given temperature or range of temperatures can be any temperature in the range of −22° C. to 40° C.

The present disclosure provides methods of stabilizing a saliva sample from a subject, the methods comprising contacting the saliva sample and a stabilization buffer of the present disclosure to produce a stabilized saliva sample.

The present disclosure provides a method of stabilizing a blood sample from a subject, the method comprising contacting the blood sample and a stabilization buffer of the present disclosure to produce a stabilized blood sample. In some aspects, the stabilization buffer and the blood sample can be contacted such that the ratio of the volume of buffer to the volume of blood sample is at least about 2 to 1. In some aspects, the stabilization buffer and the blood sample can be contacted such that the ratio of the volume of buffer to the volume of blood sample is at least about 4 to 1. In some aspects, the stabilization buffer and the blood sample can be contacted such that the ratio of the volume of buffer to the volume of blood sample is between about 2 to 1 and about 4 to 1. In some aspects, the volume of the blood sample can be at least about 35 μl, or at least about 50 or at least about 70 μl. In some aspects, the volume of the stabilization buffer can be at least about 100 μl, or at least about 140 μl, or at least about 200 μl.

In some aspects, a biological sample and a stabilization buffer of the present disclosure can be contacted in at least one sample collection tube that is pre-coated with K2-EDTA. In some aspects, a biological sample and a stabilization buffer of the present disclosure can be contacted in at least one sample collection tube that is pre-coated with K3-EDTA. In some aspects, an at least one sample collection tube can be pre-coated with at least about 10.8 mg of K2-EDTA. In some aspects, an at least one sample collection tube can be pre-coated with at least about 1 mg, or at least about 2 mg, or at least about 3 mg, or at least about 4 mg, or at least about 5 mg, or at least about 6 mg, or at least about 7 mg, or at least about 8 mg, or at least about 9 mg, or at least about 10 mg, or at least about 11 mg, or at least about 12 mg, or at least about 13 mg, or at least about 14 mg, or at least about 15 mg, or at least about 16 mg, or at least about 17 mg, or at least about 18 mg, or at least about 19 mg, or at least about 20 mg of K2-EDTA.

In some aspects, an at least one sample collection tube can be a 6 ml, 16×100 mm sample collection tube. In some aspects, the at least one sample collection tube can be a 6 ml, 16×100 mm sample collection tube that is pre-coated with K2-EDTA. In some aspects, the at least one sample collection tube can be a 6 ml, 16×100 mm sample collection tube that is pre-coated with at least about 10.8 mg of K2-EDTA. In some aspects, the at least one sample collection tube can be a 6 ml, 16×100 mm sample collection tube that is pre-coated with at least about 1 mg, or at least about 2 mg, or at least about 3 mg, or at least about 4 mg, or at least about 5 tug, or at least about 6 mg, or at least about 7 mg, or at least about 8 mg, or at least about 9 mg, or at least about 10 mg, or at least about 11 mg, or at least about 12 mg, or at least about 13 mg, or at least about 14 mg, or at least about 15 mg, or at least about 16 mg, or at least about 17 mg, or at least about 18 mg, or at least about 19 mg, or at least about 20 mg of K2-EDTA.

In some aspects, an at least one sample collection tube can be plastic. In some aspects, an at least one sample collection tube can be glass.

In some aspects, a stabilization buffer of the present disclosure and a biological sample can be contacted such that the ratio of the volume of the stabilization buffer to the volume of biological sample is at least about 2 to 1. In some aspects, a stabilization buffer of the present disclosure and a biological sample can be contacted such that the ratio of the volume of the stabilization buffer to the volume of biological sample is at least about 4 to 1. In some aspects, a stabilization buffer of the present disclosure and a biological sample can be contacted such that the ratio of the volume of the stabilization buffer to the volume of biological sample is at least about 2.2 to 1, or at least about 2.4 to 1, or at least about 2.6 to 1, or at least about 2.8 to 1, or at least about 3.0 to 1, or at least about 3.2 to 1, or at least about 3.4 to 1, or at least about 3.6 to or at least about 3.8 to 1, or at least about 4.0 to 1, or at least about 4.2 to 1, or at least about 4.4 to 1, or at least about 4.6 to 1, or at least about 4.8 to 1, or at least about 5.0 to 1. In some aspects, a stabilization buffer of the present disclosure and a biological sample can be contacted such that the ratio of the volume of the stabilization buffer to the volume of biological sample is between about 2 to 1 and about 4 to 1.

In some aspects, a stabilization buffer of the present disclosure and a blood sample can be contacted such that the ratio of the volume of the stabilization buffer to the volume of blood sample is at least about 2 to 1. In some aspects, a stabilization buffer of the present disclosure and a blood sample can be contacted such that the ratio of the volume of the stabilization buffer to the volume of blood sample is at least about 4 to 1. In some aspects, a stabilization buffer of the present disclosure and a blood sample can be contacted such that the ratio of the volume of the stabilization buffer to the volume of blood sample is at least about 2.2 to 1, or at least about 2.4 to 1, or at least about 2.6 to 1, or at least about 2.8 to 1, or at least about 3.0 to 1, or at least about 3.2 to 1, or at least about 3.4 to 1, or at least about 3.6 to 1, or at least about 3.8 to 1, or at least about 4.0 to 1, or at least about 4.2 to 1, or at least about 4.4 to 1, or at least about 4.6 to 1, or at least about 4.8 to 1, or at least about 5.0 to 1, In some aspects, a stabilization buffer of the present disclosure and a blood sample can be contacted such that the ratio of the volume of the stabilization buffer to the volume of blood sample is between about 2 to 1 and about 4 to 1.

In some aspects, a stabilization buffer of the present disclosure and a saliva sample can be contacted such that the ratio of the volume of the stabilization buffer to the volume of saliva sample is at least about 2 to 1. In some aspects, a stabilization buffer of the present disclosure and a saliva sample can be contacted such that the ratio of the volume of the stabilization buffer to the volume of saliva sample is at least about 4 to 1. In some aspects, a stabilization buffer of the present disclosure and a saliva sample can be contacted such that the ratio of the volume of the stabilization buffer to the volume of saliva sample is at least about 2.2 to 1, or at least about 2.4 to 1, or at least about 2.6 to 1, or at least about 2.8 to 1, or at least about 3.0 to 1, or at least about 3.2 to 1, or at least about 3.4 to 1, or at least about 3.6 to 1, or at least about 3.8 to 1, or at least about 4.0 to 1, or at least about 4.2 to 1, or at least about 4.4 to 1, or at least about 4.6 to 1, or at least about 4.8 to 1, or at least about 5.0 to 1. In some aspects, a stabilization buffer of the present disclosure and a saliva sample can be contacted such that the ratio of the volume of the stabilization buffer to the volume of saliva sample is between about 2 to 1 and about 4 to 1.

In some aspects, the volume of a biological sample can be at least about 35 μl. In some aspects, the volume of a biological sample can be at least about 50 μl. In some aspects, the volume of a biological sample can be at least about 2 ml. In some aspects, the volume of a biological sample can be at least about 10 μl, or at least about 15 μl, or at least about 2.0 μl, or at least about 25 μl, or at least about 30 μl, or at least about 35 μl, or at least about 40 μl, or at least about 45 μl, or at least about 50 μl, or at least about 55 μl, or at least about 60 μl, or at least about 65 μl, or at least about 70 μl, or at least about 75 μl, or at least about 80 μl, or at least about 85 μl, or at least about 90 μl, or at least about 95 μl, or at least about 100 μl, or at least about 105 μl, or at least about 110 μl, or at least about 120 μl, or at least about 130 μl, or at least about 140 μl, or at least about 150 μl, or at least about 200 μl, or at least about 500 μl, or at least about 1 ml, or at least about 1.5 ml, or at least about 2 ml, or at least about 2.5 ml, or at least about 3 ml, or at least about 3.5 ml, or at least about 4 ml, or at least about 4.5 ml, or at least about 5 ml, or at least about 6 ml, or at least about 7 ml, or at least about 8 ml, or at least about 9 ml, or at least about 10 ml.

In some aspects, the volume of a blood sample can be at least about 35 μl. In some aspects, the volume of a blood sample can be at least about 50 μl. In some aspects, the volume of a blood sample can be at least about 2 ml. In some aspects, the volume of a blood sample can be at least about 10 μl, or at least about 15 μl, or at least about 20 μl, or at least about 25 μl, or at least about 30 μl, or at least about 35 μl, or at least about 40 μl, or at least about 45 μl, or at least about 50 μl, or at least about 55 μl, or at least about 60 μl, or at least about 65 μl, or at least about 70 μl, or at least about 75 μl, or at least about 80 μl, or at least about 85 μl, or at least about 90 μl, or at least about 95 μl, or at least about 100 μl, or at least about 105 μl, or at least about 110 μl, or at least about 120 μl, or at least about 130 μl, or at least about 140 μl, or at least about 150 μl, or at least about 200 μl, or at least about 500 μl, or at least about 1 ml, or at least about 1.5 ml, or at least about 2 ml, or at least about 2.5 ml, or at least about 3 ml, or at least about 3.5 ml, or at least about 4 ml, or at least about 4.5 ml, or at least about 5 ml, or at least about 6 ml, or at least about 7 ml, or at least about 8 ml, or at least about 9 ml, or at least about 10 ml.

In some aspects, the volume of a saliva sample can be at least about 35 μl. In some aspects, the volume of a saliva sample can be at least about 50 μl. In some aspects, the volume of a saliva sample can be at least about 2 ml. In some aspects, the volume of a saliva sample can be at least about 10 μl, or at least about 15 μl, or at least about 20 μl, or at least about 25 μl, or at least about 30 μl, or at least about 35 μl, or at least about 40 μl, or at least about 45 μl, or at least about 50 μl, or at least about 55 μl, or at least about 60 μl, or at least about 65 μl, or at least about 70 μl, or at least about 75 μl, or at least about 80 μl, or at least about 85 μl, or at least about 90 μl, or at least about 95 μl, or at least about 100 μl, or at least about 105 μl, or at least about 110 μl, or at least about 120 μl, or at least about 130 μl, or at least about 140 μl, or at least about 150 μl, or at least about 200 μl, or at least about 500 μl, or at least about 1 ml, or at least about 1.5 ml, or at least about 2 ml, or at least about 2.5 nil, or at least about 3 ml, or at least about 3.5 ml, or at least about 4 ml, or at least about 4.5 ml, or at least about 5 ml, or at least about 6 ml, or at least about 7 ml, or at least about 8 ml, or at least about 9 ml, or at least about 10 ml.

In some aspects, the volume of a stabilization buffer of the present disclosure can be at least about 70 μl. In some aspects, the volume of a stabilization buffer of the present disclosure can be at least about 100 μl. In some aspects, the volume of a stabilization buffer of the present disclosure can be at least about 140 μl. In some aspects, the volume of a stabilization buffer of the present disclosure can be at least about 200 μl. In some aspects, the volume of a stabilization buffer of the present disclosure can be at least about 4 ml. In some aspects, the volume of a stabilization buffer of the present disclosure can be at least about 10 μl, or at least about 15 μl, or at least about 20 μl, or at least about 25 μl, or at least about 30 μl, or at least about 35 μl, or at least about 40 μl, or at least about 45 μl, or at least about 50 μl, or at least about 55 μl, or at least about 60 μl, or at least about 65 μl, or at least about 70 μl, or at least about 75 μl, or at least about 80 μl, or at least about 85 μl, or at least about 90 μl, or at least about 95 μl, or at least about 100 μl, or at least about 105 μl, or at least about 110 μl, or at least about 120 μl, or at least about 130 μl, or at least about 140 μl, or at least about 150 μl, or at least about 160 μl, or at least about 170 μl, or at least about 180 μl, or at least about 190 μl, or at least about 200 μl, or at least about 500 μl, or at least about 1 ml, or at least about 1.5 ml, or at least about 2 ml, or at least about 2.5 ml, or at least about 3 ml, or at least about 3.5 ml, or at least about 4 ml, or at least about 4.5 ml, or at least about 5 nil, or at least about 5.5 ml, or at least about 6.0 nil, or at least about 6.5 ml, or at least about 7.0 ml, or at least about 7.5 ml, or at least about 8.0 ml, or at least about 8.5 ml, or at least about 9 ml, or at least about 9.5 nil, or at least about 10 ml.

In some aspects of the methods of the present disclosure, after contacting a biological sample (e.g. a blood sample or a saliva sample) and a stabilization buffer, the resulting mixture is stored at about room temperature for at least about one day. In some aspects of the methods of the present disclosure, after contacting a biological sample and a stabilization buffer (e.g. a blood sample or a saliva sample), the resulting mixture is stored at about room temperature for at least about one day, or at least about two days, or at least about three days, or at least about four days, or at least about five days, or at least about six days, or at least about seven days, or at least about eight days, or at least about nine days, or at least about ten days, or at least about 11 days, or at least about 12 days, or at least about 13 days, or at least about 14 days. In some aspects, after contacting a biological sample and a stabilization buffer (e.g. a blood sample or a saliva sample), the resulting mixture is stored at about room temperature for at least about one week, or at least about two weeks, or at least about three weeks.

In some aspects, room temperature is about 22° C. in some aspects, room temperature can be about 21° C. to about 28° C. In some aspects, room temperature can be about 20° C. to about 30° C. In some aspects, room temperature can be about at least 20° C., or at least about 21° C., or at least about 22° C., or at least about 23° C., or at least about 24° C., or at least about 25° C., or at least about 26° C., or at least about 27° C., or at least about 28° C., or at least about 29° C., or at least about 30° C.

The present disclosure provides methods comprising stabilizing a biological sample from a subject by contacting the biological sample and a stabilization buffer of the present disclosure to produce a stabilized biological sample and extracting nucleic acid from the stabilized biological sample. The present disclosure provides methods comprising stabilizing a biological sample from a subject by contacting the biological sample and a stabilization buffer of the present disclosure to produce a stabilized biological sample and extracting RNA from the stabilized biological sample. The present disclosure provides methods comprising stabilizing a biological sample from a subject by contacting the biological sample and a stabilization buffer of the present disclosure to produce a stabilized biological sample and extracting DNA from the stabilized biological sample. The present disclosure provides methods comprising stabilizing a biological sample from a subject by contacting the biological sample and a stabilization buffer of the present disclosure to produce a stabilized biological sample and extracting DNA and RNA from the stabilized biological sample.

The present disclosure provides methods comprising stabilizing a blood sample from a subject by contacting the blood sample and a stabilization buffer of the present disclosure to produce a stabilized blood sample and extracting nucleic acid from the stabilized blood sample. The present disclosure provides methods comprising stabilizing a blood sample from a subject by contacting the blood sample and a stabilization buffer of the present disclosure to produce a stabilized blood sample and extracting RNA from the stabilized blood sample. The present disclosure provides methods comprising stabilizing a blood sample from a subject by contacting the blood sample and a stabilization buffer of the present disclosure to produce a stabilized blood sample and extracting DNA from the stabilized blood sample. The present disclosure provides methods comprising stabilizing a blood sample from a subject by contacting the blood sample and a stabilization buffer of the present disclosure to produce a stabilized blood sample and extracting DNA and RNA from the stabilized blood sample.

The present disclosure provides methods comprising stabilizing a saliva sample from a subject by contacting the saliva sample and a stabilization buffer of the present disclosure to produce a stabilized saliva sample and extracting nucleic acid from the stabilized saliva sample. The present disclosure provides methods comprising stabilizing a saliva sample from a subject by contacting the saliva sample and a stabilization buffer of the present disclosure to produce a stabilized saliva sample and extracting RNA from the stabilized saliva sample. The present disclosure provides methods comprising stabilizing a saliva sample from a subject by contacting the saliva sample and a stabilization buffer of the present disclosure to produce a stabilized saliva sample and extracting DNA from the stabilized saliva sample. The present disclosure provides methods comprising stabilizing a saliva sample from a subject by contacting the saliva sample and a stabilization buffer of the present disclosure to produce a stabilized saliva sample and extracting DNA and RNA from the stabilized saliva sample.

In some aspects, nucleic acids, including RNA, DNA or RNA and DNA can be extracted from a stabilized blood sample using nucleic acid extraction methods that are known in the art. In a non-limiting example, RNA can be extracted from a stabilized blood sample using methods comprising the use of TRIzol LS. In a non-limiting example, RNA can be extracted from a stabilized blood sample using methods comprising the use of TRIzol LS and chloroform. In a non-limiting example, RNA can be extracted from a stabilized blood sample using methods comprising beads (magnetic or silica-coated). In a non-limiting example, RNA can be extracted from a stabilized blood sample using methods comprising the use of phenol. In some aspects, RNA can be extracted from a stabilized blood sample using commercially available kits, including, but not limited to, Qiagen PAXgene Blood miRNA Kit, Thermo Fisher MagMAX for Stabilized Blood Tubes RNA Isolation Kit, and Norgen Biotek Preserved Blood RNA Purification Kit I and Kit II, Qiagen QIAsymphony, Thermo Fisher MagMAX Express-96 Magnetic Particle Processor, RNAqueous kit (Thermo Fisher), Micro-to-midi total RNA purification system (Thermo Fisher), NucleoSpin RNA II (Becton and Dickinson), GenElute mammalian total RNA kit (MilliporeSigma), RNeasy mini kit (Qiagen), and TRIzol LS reagent (Thermo Fisher).

In some aspects, extracting RNA from a stabilized biological sample (e.g. blood sample or saliva sample) can comprise contacting the stabilized biological sample with TRIzol LS. In some aspects, a stabilized biological sample can be diluted prior to contact with TRIzol LS.

In a non-limiting example, a 150 μl stabilized biological sample can be diluted to a total volume of 250 μl with 100 μl or RNase free water prior to the addition of 750 μl of TRIzol LS. After a stabilized biological sample or a diluted stabilized biological sample is contacted with TRIzol LS, the resulting mixture can be vortexed and incubated at room temperature. In a non-limiting example, after a stabilized biological sample or a diluted stabilized biological sample is contacted with TRIzol LS, the resulting mixture can be vortexed for at least about 10 seconds and incubated at room temperature for at least about 5 minutes. Following vortexing and incubation at room temperature, the mixture can be contacted with chloroform. In a non-limiting example, at least about 200 μl of chloroform can be added for each milliliter of Trizol LS-stabilized blood sample mixture. After contacting with chloroform, the resulting mixture can be vortexed. In a non-limiting example, the resulting mixture can be vortexed for at least about 15 seconds. After vortexing, the mixture can be incubated at room temperature. In a non-limiting example, the mixture can be incubated at room temperature for at least about 3 minutes. After incubation at room temperature, the mixture can be centrifuged. In a non-limiting example, the mixture can be centrifuged at 13,000×g at about 4° C. for at least about 25 minutes. After centrifugation, the aqueous phase (typically yellow to clear in color, located on top) can be transferred to a new sample tube. The transferred aqueous phase can then be contacted with isopropanol. In a non-limiting example, the transferred aqueous phase can be contacted with at least about 500 μl of isopropanol. After contact with isopropanol, the resulting mixture can be incubated at room temperature. In a non-limiting example, the resulting mixture can be incubated at room temperature for at least about 10 minutes. After incubation at 10 minutes, the mixture can be centrifuged to yield a pellet. In a non-limiting example, the mixture can be centrifuged at 13,000×g at about 4° C. for at least about 10 minutes. After centrifugation, the resulting supernatant can be removed from the pellet. 70% ethanol can then be added to the pellet. In a non-limiting example, at least about 1000 μl of 70% ethanol can be added to the pellet. The pellet can then be resuspended in the added 70% ethanol. After resuspending the pellet, the resulting mixture can be centrifuged. In a non-limiting example, the resulting mixture can be centrifuged at 10,000×g at about 4° C. for at least about 5 minutes to yield a pellet. After centrifugation, the supernatant can be removed and the pellet allowed to dry for at least about 10 minutes. After drying, the pellet can be resuspended with at least about 100 μl of RNase free water and allowed to dissolve at room temperature. The RNA can then further be purified using techniques standard in the art, including, but not limited to, the use of commercially available kits such as Qiagen RNeasy Mini Kit.

In some aspects, the methods of the present disclosure can further comprise, prior to contacting the stabilized biological sample with at least one agent specific to detect the expression of at least one biomarker, extracting RNA from the stabilized biological sample. In some aspects, the methods of the present disclosure can further comprise, prior to contacting the stabilized biological sample with at least one agent specific to detect the expression of at least one biomarker, extracting nucleic acid from the stabilized biological sample. In some aspects, the methods of the present disclosure can further comprise, prior to contacting the stabilized biological sample with at least one agent specific to detect the expression of at least one biomarker, extracting DNA from the stabilized biological sample. In some aspects, RNA can be extracted using a method comprising contacting a stabilized biological sample and TRIzol LS. In some aspects, a stabilized biological sample can be diluted prior to contacting the stabilized biological sample and TRIzol LS. In some aspects, after contacting a stabilized biological sample and TRIzol LS, the resulting mixture can be contacted with chloroform.

The present disclosure provides prognostic, predictive, diagnostic and therapeutic methods, the methods comprising: i) extracting RNA from a biological sample stabilized using a method and/or composition of the present disclosure; ii) determining the expression level of at least one RNA transcript in the extracted RNA; and iii) diagnosing the subject with a disease and/or disorder, providing a treatment recommendation to the subject, monitoring the progression of a disease and/or disorder in the subject, or administering at least one therapeutic to the subject based on the expression level of the at least one RNA transcript.

In some aspects, determining the expression level of the at least one RNA transcript can comprise the use of quantitative PCR. As would be appreciated by the skilled artisan, any quantitative PCR method known in the art can be used to determine the expression level of the at least one RNA transcript.

In some aspects, prior to the use of quantitative PCR, the at least one RNA transcript can be reverse transcribed to produce cDNA, and the expression level of the cDNA can be measured using quantitative PCR. As would be appreciated by the skilled artisan, any reverse-transcription technique standard in the art can be used.

In some aspects, prior to the use of quantitative PCR, the at least one RNA transcript can be reverse transcribed to produce cDNA, then the cDNA can be amplified and then the expression level of the amplified cDNA can be measured using quantitative PCR. As would be appreciated by the skilled artisan, any nucleic amplification techniques (e.g. selective PCR) and any reverse-transcription technique standard in the art can be used.

In some aspects, the disease and/or disorder can be cancer, gastroenteropancreatic (GEP) neuroendocrine neoplasm (GEP-NEN), melanoma, multiple myeloma, a plasma cell dyscrasia, monoclonal gammopathy of undetermined significance (MGUS), colon cancer, prostate cancer or SARS-CoV-2 infection.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, leukemia and germ cell tumors. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma. Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer, cervical cancer, Acute lymphoblastic leukemia (ALL), Acute myeloid leukemia (AML), Adrenal gland tumors, Anal cancer, Bile duct cancer, Bladder cancer, Bone cancer, Bowel cancer, Brain tumors, Breast cancer, Cancer of unknown primary (CUP), Cancer spread to bone, Cancer spread to brain, Cancer spread to liver, Cancer spread to lung, Carcinoid, Cervical cancer, Children's cancers, Chronic lymphocytic leukemia (CLL), Chrome myeloid leukemia (CMI), Colorectal cancer, Ear cancer, Endometrial cancer, Eye cancer, Follicular dendritic cell sarcoma, Gallbladder cancer, Gastric cancer, Gastro esophageal junction cancers, Germ cell tumors, Gestational trophoblastic disease (GIT)), Hairy cell leukemia, Head and neck cancer, Hodgkin lymphoma, Kaposi's sarcoma, Kidney cancer, Laryngeal cancer, Leukemia, Gastric linitis plastica, Liver cancer, Lung cancer, Lymphoma, Malignant schwannoma, Mediastinal germ cell tumors, Melanoma skin cancer, Men's cancer, Merkel cell skin cancer, Mesothelioma, Molar pregnancy, Mouth and oropharyngeal cancer, Myeloma, Nasal and paranasal sinus cancer, Nasopharyngeal cancer, Neuroblastoma, Neuroendocrine tumors, Non-Hodgkin lymphoma (NHL), Esophageal cancer, Ovarian cancer, Pancreatic cancer, Penile cancer, Persistent trophoblastic disease and choriocarcinoma, Pheochromocytoma, Prostate cancer, Pseudomyxoma peritonei, Rectal cancer. Retinoblastoma, Salivary gland cancer, Secondary’ cancer, Signet cell cancer, Skin cancer, Small bowel cancer, Soft tissue sarcoma, Stomach cancer, T cell childhood non Hodgkin lymphoma (NHL), Testicular cancer, Thymus gland cancer, Thyroid cancer, Tongue cancer, Tonsil cancer, Tumors of the adrenal gland, Uterine cancer. Vaginal cancer, Vulval cancer, Wilms' tumor, Womb cancer and Gynaecological cancer, Examples of cancer also include, but are not limited to, Hematologic malignancies, Lymphoma, Cutaneous T-cell lymphoma, Peripheral T-cell lymphoma, Hodgkin's lymphoma, Non-Hodgkin's lymphoma, Multiple myeloma, Chrome lymphocytic leukemia, chronic myeloid leukemia, acute myeloid leukemia, Myelodysplastic syndromes, Myelofibrosis, Biliary tract cancer, Hepatocellular cancer, Colorectal cancer, Breast cancer, Lung cancer, Non-small cell lung cancer, Ovarian cancer, Thyroid Carcinoma, Renal Cell Carcinoma, Pancreatic cancer, Bladder cancer, skin cancer, malignant melanoma, merkel cell carcinoma, Uveal Melanoma or Glioblastoma multiforme.

In some aspects, the stabilization compositions, methods and kits of the present disclosure can be used in combination with the prognostic, predictive, diagnostic and therapeutic methods related to the detection and treatment of Gastroenteropancreatic (GEP) neuroendocrine neoplasm (GEP-NEN), also referred to as Gastroenteropancreatic Neuroendocrine Tumor and Neuroendocrine Tumor (NET). In some aspects, the stabilization compositions, methods and kits of the present disclosure can be used in combination with the prognostic, predictive, diagnostic and therapeutic methods described in WO2012/119013, U.S. Pat. No. 9,988,684, WO2016/044330 and U.S. Pat. No. 10,407,730, the contents of which are incorporated in their entities. These prognostic, predictive, diagnostic and therapeutic methods can include the calculation of a NETest score, which, as would be appreciated by the skilled artisan, refers to the output of a mathematically-derived classifier algorithm generated from the combination of classification algorithms, i.e. SVM, LDA, KNN, and Bayes. This score ranges between 0 and 100%. The expression level score from a test sample, once compared to the expression level score for a reference or control sample, may be used to diagnose the presence of GEP-NEN, the different stages of GEP-NEN, predict the risk of contracting a stage of GEP-NEN, or determines the risk of recurrence of GEP-NEN in post-therapy human patients. Distinctions between GEP-NEN disease states are based on pre-determined expression level score thresholds and/or ranges as further defined in the present application (see WO/2012/119013, U.S. Pat. No. 9,988,684, WO/2016/044330 and U.S. Pat. No. 10,407,730;

In some aspects, the stabilization compositions, methods and kits of the present disclosure can be used in combination with the prognostic, predictive, diagnostic and therapeutic methods related to melanoma. In some aspects, the stabilization compositions, methods and kits of the present disclosure can be used in combination with the prognostic, predictive, diagnostic and therapeutic methods described in WO2018/217627 and US 2018-0340934 A1, the contents of which are incorporated in their entities.

In some aspects, the stabilization compositions, methods and kits of the present disclosure can be used in combination with the prognostic, predictive, diagnostic and therapeutic methods related to multiple myeloma, a plasma cell dyscrasia, monoclonal gammopathy of undetermined significance (MGUS) or any other related disease/disorder. In some aspects, the stabilization compositions, methods and kits of the present disclosure can be used in combination with the prognostic, predictive, diagnostic and therapeutic methods described in WO2019/018540 and US 2019-0025311 A1, the contents of which are incorporated in their entities.

In some aspects, the stabilization compositions, methods and kits of the present disclosure can be used in combination with the prognostic, predictive, diagnostic and therapeutic methods related to predicting response to peptide receptor radiotherapy (PRRT). In some aspects, the stabilization compositions, methods and kits of the present disclosure can be used in combination with the prognostic, predictive, diagnostic and therapeutic methods described in WO2019/108734 and US 2019-0160189 A1, the contents of which are incorporated in their entities.

In some aspects, the stabilization compositions, methods and kits of the present disclosure can be used in combination with the prognostic, predictive, diagnostic and therapeutic methods related to colon cancer. In some aspects, the stabilization compositions, methods and kits of the present disclosure can be used in combination with the prognostic, predictive; diagnostic and therapeutic methods described in WO 2019/144099 and US 2019-0226030 A1, the contents of which are incorporated in their entities.

In some aspects, the stabilization compositions, methods and kits of the present disclosure can be used in combination with the prognostic, predictive, diagnostic and therapeutic methods related to prostate cancer. In some aspects, the stabilization compositions, methods and kits of the present disclosure can be used in combination with the prognostic, predictive, diagnostic and therapeutic methods described in WO 2019/165021 and US 2019-0259471 A1, the contents of which are incorporated in their entities.

In some aspects, the stabilization compositions, methods and kits of the present disclosure can be used in combination with prognostic, predictive, diagnostic and therapeutic methods related to SARS-CoV-2 infection. As would be appreciated by the skilled artisan, SARS-CoV-2 is the virus responsible for Coronavirus disease 2019 (COVID-19). In some aspects, the stabilization compositions, methods and kits of the present disclosure can be used to stabilize saliva samples for subsequent extraction of RNA and analysis for levels of SARS-CoV-2 RNA.

In some aspects, the present disclosure provides methods of identifying SARS-CoV-2 infection in a subject, the methods comprising: (a) obtaining a saliva sample from the subject; (b) contacting the saliva sample with at least one stabilization buffer of the present disclosure, thereby producing a stabilize saliva sample; (c) extracting RNA from the stabilized saliva sample; (d) determining the expression level of at least one SARS-CoV-2 specific transcript in the extracted RNA; (e) comparing the expression level of the at least one SARS-CoV-2 specific transcript to a predetermined cutoff value; and (f) determining that the subject has a SARS-CoV-2 infection when the expression level of the at least one SAM-CoV-2 specific transcript is greater than or equal to the predetermined cutoff value.

In some aspects, the at least one SARS-CoV-2 specific transcript comprises a fragment of the SARS-CoV-2 genome (SEQ ID NO: 1). In some aspects, the at least one SARS-CoV-2 specific transcript, comprises the portion of the SARS-CoV-2 genome encoding the nucleocapsid “N” (SEQ ID NO: 2). In some aspects, the at least one SARS-CoV-2 specific transcript comprises a fragment of the portion of the SARS-CoV-2 genome encoding the nucleocapsid “N”. In some aspects, a fragment of the portion of the SARS-CoV-2 genome encoding the nucleocapsid protein can comprises, consist essentially of, or consist of the nucleotide sequence: GACCCCAAAATCAGCGAAATGCACCCCGCATTACGTTTGGTGGACCCTCAGATTCAACTGGCAGTAACCAGA (SEQ ID NO: 3), hereafter referred to as “N1”. In some aspects a fragment of the portion of the SARS-CoV-2 genome encoding the nucleocapsid protein can comprise, consist essentially of, or consist of the nucleotide sequence: TTACAAACATTGGCCGCAAATTGCACAATTTGCCCCCAGCGCTTCAGCGTTCTTCGGAATGTCGCGC (SEQ ID NO: 4), hereafter referred to as “N3”.

In some aspects of the preceding method, determining the expression level of at least one SARS-CoV-2 specific transcript in the extracted RNA comprises determining the expression level of N1, In some aspects of the preceding method, determining the expression level of at least one SARS-Cob′-2 specific transcript in the extracted RNA comprises determining the expression of N3. In some aspects of the preceding method, determining the expression level of at least one SARS-CoV-2 specific transcript in the extracted RNA comprises determining the expression level of N1 and N3.

In some aspects the preceding method further comprises measuring the expression level of at least one positive control gene. In some aspects, the positive control gene can be spiked into a sample at any step of the method. In some aspects, the positive control gene is a control plasmid comprising at least one SARS-CoV-2 specific gene.

In some aspects, the preceding method further comprises measuring the expression level of at least one quality control gene. Without wishing to be bound by theory, the expression level of the quality-control gene can be used to determine if the saliva sample was appropriately collected and processed. In some aspects, if the expression level of the quality control gene is too high (i.e. greater than a predetermined cutoff value), the sample can be deemed to have been processed incorrectly and therefore discarded. In some aspects, the at least one quality control gene is an RNase. In some aspects, the at least one quality control gene is RNase P (chromosome 10 (10q23.31); UniGene ID: Hs.139120; RefSeq: NC_000010.11).

In some aspects of the preceding method, determining the expression level of at least one SARS-CoV-2 specific transcript in the extracted RNA can comprise using quantitative PCR to measure the expression level of the at least one transcript in the extracted RNA, using standard quantitative PCR techniques known in the art. In some aspects of the preceding method, determining the expression level of at least one SARS-CoV-2 specific transcript in the extracted RNA can comprise reverse transcribing the extracted RNA to form cDNA using techniques standard in the art, as would be appreciated by the skilled artisan. The expression level of the at least SARS-CoV-2 specific transcript in the cDNA can then be quantified using quantitative PCR, as would be appreciated by the skilled artisan.

In aspects of the preceding method wherein the expression level of N1 is determined using quantitative PCR, at least one of the SARS-CoV-2_N1 Forward Amplification Primer, SARS-CoV-2_N1 Reverse Amplification Primer, and the SARS-CoV-2_N1 Detection Probe presented in Table 1 can be used in the quantitative PCR reaction.

In aspects of the preceding method wherein the expression level of N1 is determined using quantitative PCR, at least one of the SARS-CoV-2_N3 Forward Amplification Primer, SARS-CoV-2_N3 Reverse Amplification Primer, and the SARS-CoV-2_N3 Detection Probe presented in Table 1 can be used in the quantitative PCR reaction.

In aspects of the preceding method wherein the expression level of RNAse P is determined using quantitative PCR, at least one of the RNAse P Forward Amplification Primer, RNAse P Reverse Amplification Primer and the RNAse P Detection Probe presented in Table 1 can be used in the quantitative PCR reaction.

TABLE 1

| | | | SEQ |
|---|---|---|---|
| | | | ID |
| Description | Oligonucleotide Sequence (5′>3′) | Label | NO: |
| SARS-CoV-2_N1 | 5′-GAC CCC AAA ATC AGC GAA AT-3′ | None | 5 |
| Forward | | | |
| Amplification Primer | | | |
| SARS-CoV-2_N1 | 5′-TCT GGT TAC TGC CAG TTG AAT CTG-3′ | None | 6 |
| Reverse | | | |
| Amplification Primer | | | |
| SARS-CoV-2_N1 | 5′-FAM-ACC CCG CAT TAC GTT TGG ACC-3′ | FAM | 7 |
| Detection | | | |
| Probe | | | |
| SARS-CoV-2_N3 | 5′-GGG AGC CTT GAA TAC ACC AAA A-3′ | None | 8 |
| Forward | | | |
| Amplification Primer | | | |
| SARS-CoV-2_N3 | 5′-TGT AGC ACG ATT GCA TTG-3′ | None | 9 |
| Reverse | | | |
| Amplification Primer | | | |
| SARS-CoV-2_N3 | 5′-FAM-ATC ACA TTG GCA CCC GCA ATC CTG-3′ | FAM | 10 |
| Detection Probe | | | |
| RNAse P Forward | 5′-AGA TTT GGA CCT GCG AGC G-3′ | None | 11 |
| Amplification Primer | | | |
| RNAse P Reverse | 5′-GAG CGG CTG TCT CCA CAA GT-3′ | None | 12 |
| Amplification Primer | | | |
| RNAse P Detection | 5′-FAM-TTC TGA CCT GAA GGC TCT GCG CG-3′ | FAM | 13 |
| Probe | | | |

Kits

The present disclosure provides kits comprising at least one amount of at least one stabilization buffer of the present disclosure. In some aspects, the kits can further comprise at least on sample collection tube. In some aspects, the at least one amount of the at least one stabilization buffer of the present disclosure can be contained in the sample tube.

In some aspects, the kits of the present disclosure can further comprise at least one plurality of at least one amplification primer. In a non-limiting example, the amplification primer can be any one of the amplification primers recited in Table 1. In some aspects, the kits of the present disclosure can comprise a plurality of each of the amplification primers recited in Table 1. In some aspects, the kits of the present can further comprise at least one plurality of at least one probe. In a non-limiting example, the probe can be any one of the probes recited in Table 1. In some aspects, the kits of the present disclosure can comprise a plurality of each of the probes recited in Table 1.

The present disclosure provides a kit comprising at least one sample collection tube, wherein the at least one sample collection tube comprises at least about 70 μl of a stabilization buffer of the present disclosure. The present disclosure provides a kit comprising at least one sample collection tube, wherein the at least one sample collection tube comprises at least about 100 μl of a stabilization buffer of the present disclosure. The present disclosure provides a kit comprising at least one sample collection tube, wherein the at least one sample collection tube comprises at least about 4 ml of a stabilization buffer of the present disclosure.

The present disclosure provides a kit for stabilizing at least one biological sample at room temperature for at least one day comprising at least one sample collection tube, wherein the at least one sample collection tube comprises at least about 70 μl of a stabilization buffer of the present disclosure. The present disclosure provides a kit for stabilizing at least one biological sample at room temperature for at least one day comprising at least one sample collection tube, wherein the at least one sample collection tube comprises at least about 100 μl of a stabilization buffer of the present disclosure. The present disclosure provides a kit for stabilizing at least one biological sample at room temperature for at least one day comprising at least one sample collection tube, wherein the at least one sample collection tube comprises at least about 4 ml of a stabilization buffer of the present disclosure.

The present disclosure provides a kit for stabilizing at least one blood sample at room temperature for at least one day comprising at least one sample collection tube, wherein the at least one sample collection tube comprises at least about 70 μl of a stabilization buffer of the present disclosure. The present disclosure provides a kit for stabilizing at least one blood sample at room temperature for at least one day comprising at least one sample collection tube, wherein the at least one sample collection tube comprises at least about 100 μl of a stabilization buffer of the present disclosure. The present disclosure provides a kit for stabilizing at least one blood sample at room temperature for at least one day comprising at least one sample collection tube, wherein the at least one sample collection tube comprises at least about 4 ml of a stabilization buffer of the present disclosure.

The present disclosure provides a kit comprising at least one sample collection tube, wherein the at least one sample collection tube comprises at least about 10 μl, or at least about 15 μl, or at least about 20 μl, or at least about 25 μl, or at least about 30 μl, or at least about 35 μl, or at least about 40 μl, or at least about 45 μl, or at least about 50 μl, or at least about 55 μl, or at least about 60 μl, or at least about 65 μl, or at least about 70 μl, or at least about 75 μl, or at least about 80 μl, or at least about 85 μl, or at least about 90 μl, or at least about 95 μl, or at least about 100 μl, or at least about 105 μl, or at least about 110 μl, or at least about 120 μl, or at least about 130 μl, or at least about 140 μl, or at least about 150 μl, or at least about 160 μl, or at least about 170 μl, or at least about 180 μl, or at least about 190 μl, or at least about 200 μl, or at least about 500 μl, or at least about 1 ml, or at least about 1.5 ml, or at least about 2 ml, or at least about 2.5 ml, or at least about 3 ml, or at least about 3.5 ml, or at least about 4 ml, or at least about 4.5 ml, or at least about 5 ml, or at least about 5.5 ml, or at least about 6.0 ml, or at least about 6.5 ml, or at least about 7.0 ml, or at least about 7.5 ml, or at least about 8.0 ml, or at least about 8.5 ml, or at least about 9 ml, or at least about 9.5 ml, or at least about 10 ml of a stabilization buffer of the present disclosure.

In some aspects, an at least one sample collection tube can be a 6 ml, 16×100 mm sample collection tube. In some aspects, the at least one sample collection tube can be a 6 ml, 16×100 mm sample collection tube that is pre-coated with K2-EDTA. In some aspects, the at least one sample collection tube can be a 6 ml, 16×100 mm sample collection tube that is pre-coated with at least about 10.8 mg of K2-EDTA. In some aspects, the at least one sample collection tube can be a 6 ml, 16×100 mm sample collection tube that is pre-coated with at least about 1 mg, or at least about 2 mg, or at least about 3 mg, or at least about 4 mg, or at least about 5 mg, or at least about 6 mg, or at least about 7 mg, or at least about 8 mg, or at least about 9 mg, or at least about 10 mg, or at least about 11 mg, or at least about 12 mg, or at least about 13 mg, or at least about 14 mg, or at least about 15 mg, or at least about 16 mg, or at least about 17 mg, or at least about 18 mg, or at least about 19 mg, or at least about 20 mg of K2-EDTA.

In some aspects, an at least one sample collection tube can be plastic. In some aspects, an at least one sample collection tube can be glass. In some aspects, an at least one sample collection tube can be sealed. In some aspects, an at least one sample collection tube can have been processed such that the air has been evacuated out if the at least one sample collection tube.

In some aspects, a kit of the present disclosure can comprise instructions for use. The instructions can be written instructions. A kit of the present disclosure can be used for any method described herein.

In some aspects, a kit of the present disclosure can comprise at least about 1 agent, or at least about 2, or at least about 3, or at least about 4, or at least about 5, or at least about 6, or at least about 7, or at least about 8, or at least about 9, or at least about 10, or at least about 11, or at least about 12, or at least about 13, or at least about 14, or at least about 15, or at least about 16, or at least about 17, or at least about 18, or at least about 19, or at least about 20, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39, or at least about 40 agents specific to detect the expression of at least about one biomarker, or at least about 2, or at least about 3, or at least about 4, or at least about 5, or at least about 6, or at least about 7, or at least about 8, or at least about 9, or at least about 10, or at least about 11, or at least about 12, or at least about 13, or at least about 14, or at least about 15, or at least about 16, or at least about 17, or at least about 18, or at least about 19, or at least about 20, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39, or at least about 40 biomarkers. In some aspects, an agent specific to detect the expression of at least one biomarker can comprise a primer, a pair of primers, a sense and anti-sense primer pair, a polynucleotide that specifically, hybridizes to a biomarker or any combination thereof.

In some aspects of the methods of the present disclosure, a biomarker can be RNA, cDNA, or protein. In some aspects of the methods of the present disclosure, wherein the biomarker is RNA, the RNA can be reverse transcribed to produce cDNA, and the produced cDNA expression level can be detected. In some aspects, the expression level of a biomarker can be detected by forming a complex between the biomarker and a labeled probe or primer. In some aspects of the methods of the present disclosure, wherein a biomarker is a protein, the protein can be detected by forming a complex between the protein and a labeled antibody. In some aspects of the present disclosure, wherein a biomarker is RNA and/or cDNA, the RNA and/or cDNA can be detected by forming a complex between the RNA and/or cDNA and a labeled nucleic acid probe or primer. A complex between the RNA or cDNA and the labeled nucleic acid probe or primer can be a hybridization complex. In some aspects, a label can be a fluorescent label.

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore the term diagnosis includes: a. prediction (determining if a patient will likely develop aggressive disease (hyperproliferative/invasive)), b. prognosis (predicting whether a patient will likely have a better or worse outcome at a pre-selected time in the future), c. therapy selection, d. therapeutic drug monitoring, and e. relapse monitoring.

The term “subject” as used herein refers to a mammal, preferably a human.

“Treating” or “treatment” as used herein with regard to a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

Biomarker levels may change due to treatment of the disease. The changes in biomarker levels may be measured by the methods of the present disclosure. Changes in biomarker levels may be used to monitor the progression of disease or therapy.

“Altered”, “changed” or “significantly different” refer to a detectable change or difference from a reasonably comparable state, profile, measurement, or the like. Such changes may be all or none. They may be incremental and need not be linear. They may be by orders of magnitude. A change may be an increase or decrease by 1%, 5%, 10%, 20%, 30%, 40%, 50%, 60%, 70%, 80%, 90%, 95%, 99%, 100%, or more, or any value in between 0% and 100%. Alternatively, the change may be 1-fold, 1.5-fold 2-fold, 3-fold, 4-fold, 5-fold or more, or any values in between 1-fold and five-fold. The change may be statistically significant with a p value of 0.1, 0.05, 0.001, or 0.0001.

As used herein, the terms “expression level” and “amount” are used interchangeably to refer to the amount of a specific molecule (e.g. a specific RNA transcript) present in a given biological sample or from biological material extracted form a biological sample.

### EXAMPLES

### Example 1—Stabilizing Blood and Blood RNA Using the Stabilization Buffers and Methods of the Present Disclosure

The following non-limiting example demonstrates that the stabilization buffers and methods of the present disclosure can effectively stabilize biological samples, such as blood samples. Blood was collected by venipuncture from four neuroendocrine tumor patients and then either stored immediately at −80° C. per standard protocol in an EDTA-coated tube or collected into stabilization buffer at a ratio of 1:2, sample volume to buffer volume. The stabilization buffer comprised guanidine hydrochloride at a concentration of at least about 4.5 M, Triton X-100 at a concentration of at least about 0.1% (volume/volume), EDTA at a concentration of at least about 20 mM, citric acid at a concentration of at least about 80 mM; and sodium citrate at a concentration of at least about 120 mM. The pH of the stabilization buffer was between about 4.05 and about 4.11. The samples were then incubated at room temperature (21° C.-28° C.) for 1, 2 or 3 days. The concentration and quality of RNA isolated was evaluated, as was the number of genes amplified by PCR, and the neuroendocrine tumor score (NETest) was calculated for each sample using the methods of the present disclosure.

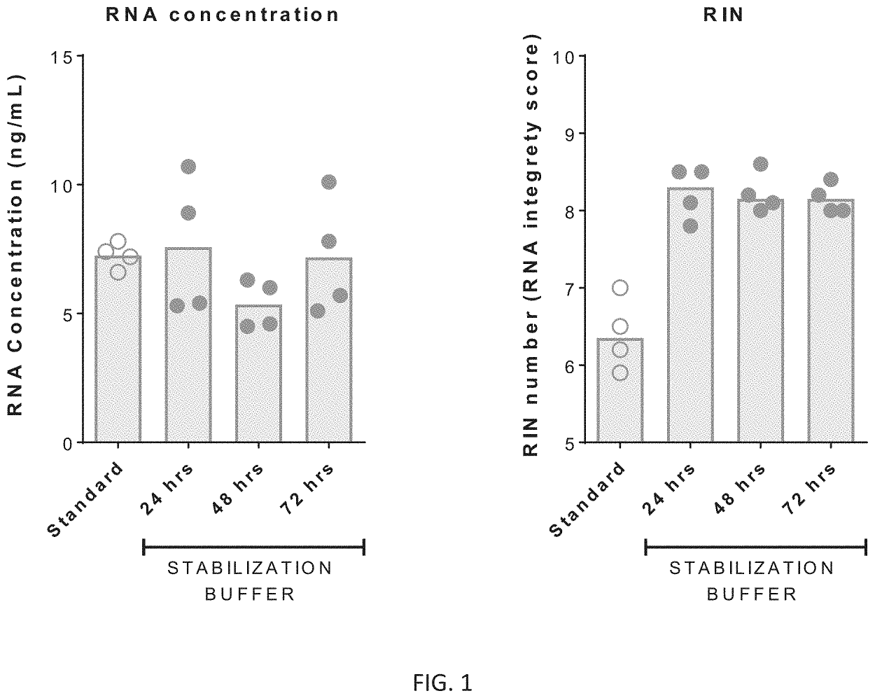

As shown in the left panel of FIG. 1, RNA concentration levels collected per standard protocol and frozen at −80° C. before isolation (7.2±0.3 ng/mL) were similar to levels after 24 hrs stabilization at room temperature (7.6±1.3 ng/mL), 48 hrs stabilization (5.3±0.5 ng/mL) or 72 hrs stabilization (7.2±1.1 ng/mL). As shown in the right panel of FIG. 1, the RNA integrity numbers (RIN) were significantly higher (p<0.05) in stabilization blood (mean: 8.2) than standard blood collection (6.4±0.2).

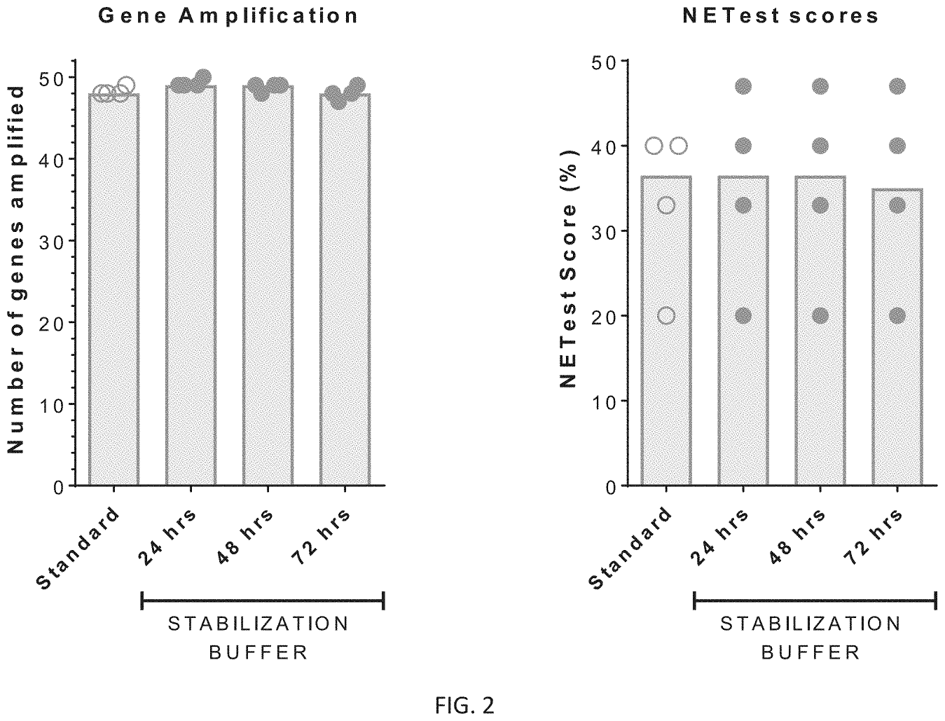

As shown in the left panel of FIG. 2, gene amplification was similar in both the standard collection samples (48-49) and the stabilized samples (47-50). As shown in the right panel of FIG. 2, NETest scores, were not significantly different between the standard collection samples (33±4.7) and the stabilized samples (35±6).

These results demonstrate that the stabilization buffers and methods of the present disclosure can effectively stabilize blood samples. In fact, the stabilization buffers and methods of the present disclosure preserve the blood samples to a greater extent than the existing standard protocol of freezing at −80° C.

### Example 2—Gene Expression and NETest Scores Measured Using Different Volumes of Stabilized Blood

The following non-limiting example demonstrates that the stabilization buffers and methods of the present disclosure can effectively stabilize biological samples, such as blood samples. Blood was collected from five neuroendocrine tumor patients and four control patients via venipuncture. The blood samples were collected directly into the stabilization buffer of example 1. The stabilized blood samples were then incubated at room temperature (21° C.-28° C.) for 72 hours. The gene expression and NETest scores in different blood volumes ranging between 5 and 50 μl of stabilized blood was then measured and analyzed.

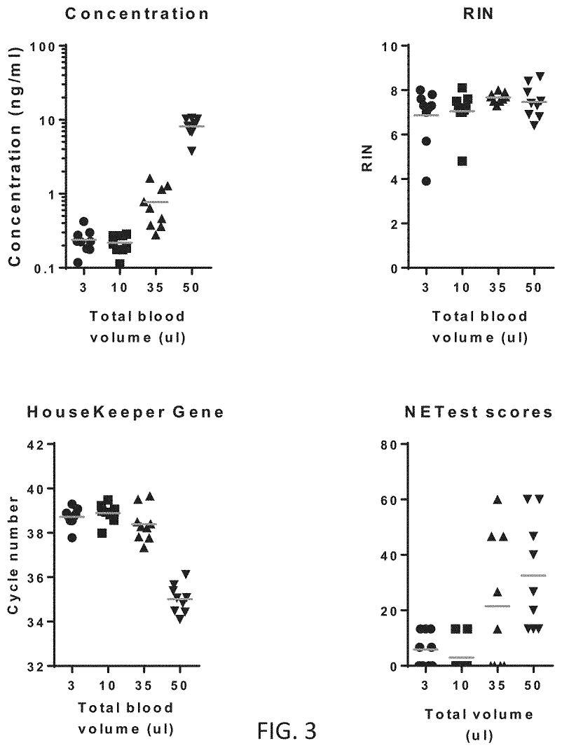

As shown in the top left panel of FIG. 3, RNA concentration levels ranged from 0.2-0.8 ng/ml (5-35 μl) and were significantly (p<0.0001) higher in 50 μl samples. As shown in the top right panel of FIG. 3, RIN values ranged from 6.8 to 7.6 and were not significantly different between samples. As shown in the bottom left panel of FIG. 3, target gene expression (cycle number) was ˜38 for 5-35 μl. At 50 μl, the cycle time was significantly lower (consistent with higher gene expression)−35±0.2 (p<0.0005). As show in the bottom right panel of FIG. 3, the NETest score ranged from 5.9±2 (5 μl of stabilized blood) to 32.6±6.5 (50 μl stabilized blood).

These results indicate that various volumes, including volumes ranging from 5 to 50 μl of blood stabilized using the methods and compositions of the present disclosure can be used in subsequent diagnostic, prognostic and therapeutic applications.

### Example 3—RNA Concentration and NETest Scores Measured Using Different Volumes of Blood Collected by Venipuncture or Finger Prick Stabilized Using the Methods and Compositions of the Present Disclosure

The following non-limiting example demonstrates that the stabilization buffers and methods of the present disclosure can effectively stabilize biological samples, such as blood samples, at varying sample volumes. Blood was collected via venipuncture or finger prick directly into the stabilization buffer of Example 1 from seven neuroendocrine tumor patients. The stabilized blood samples were then incubated at room temperature (21° C.-28° C.) for 72 hours. Sample sizes of 35 μl and 50 μl of blood were collected by finger prick and a sample size of 50 μl of blood was collected by venipuncture. The concentration and quality of RNA isolated was evaluated in each stabilized sample, as was the individual gene expression and the neuroendocrine tumor score (NETest).

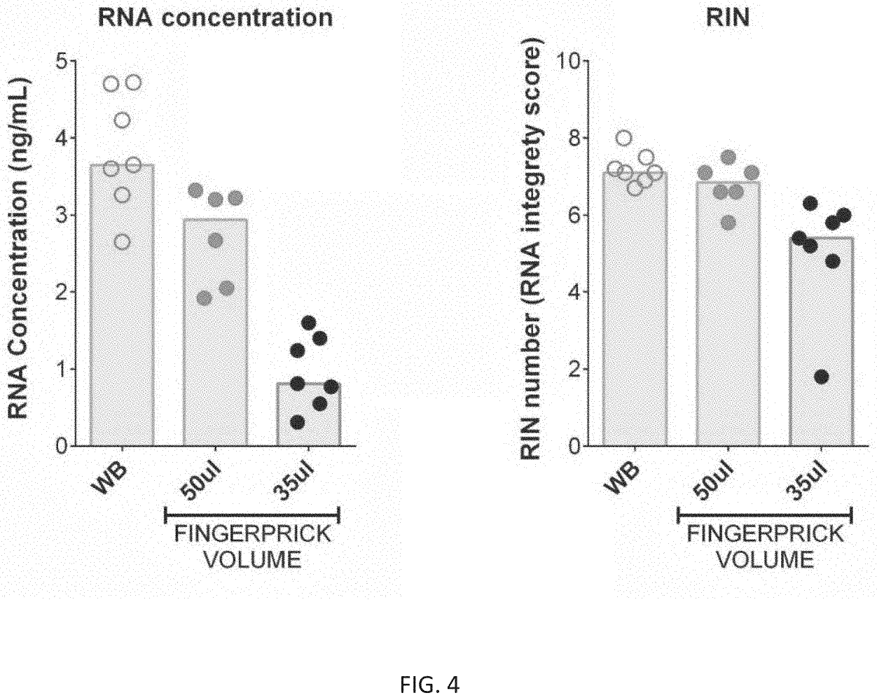

As shown in the left panel of FIG. 4, RNA concentration levels from stabilized whole blood/venipuncture samples (3.8±0.8 ng/mL) were similar to levels from the stabilized 50 μl fingerprick samples (2.7±0.8 ng/mL). Levels in the stabilized 35 μl fingerprick samples (0.95±0.47 ng/mL) were significantly (p<0.03) lower. As shown in the right panel of FIG. 4, the RNA integrity numbers (RIN) were similar between the stabilized whole blood samples (7.2±0.4 ng/mL) and the stabilized 50 μl fingerprick samples (6.8±0.6 ng/mL), but were significantly lower in the stabilized 35 μl fingerprick samples (5±1.5 ng/mL, p<0.03).

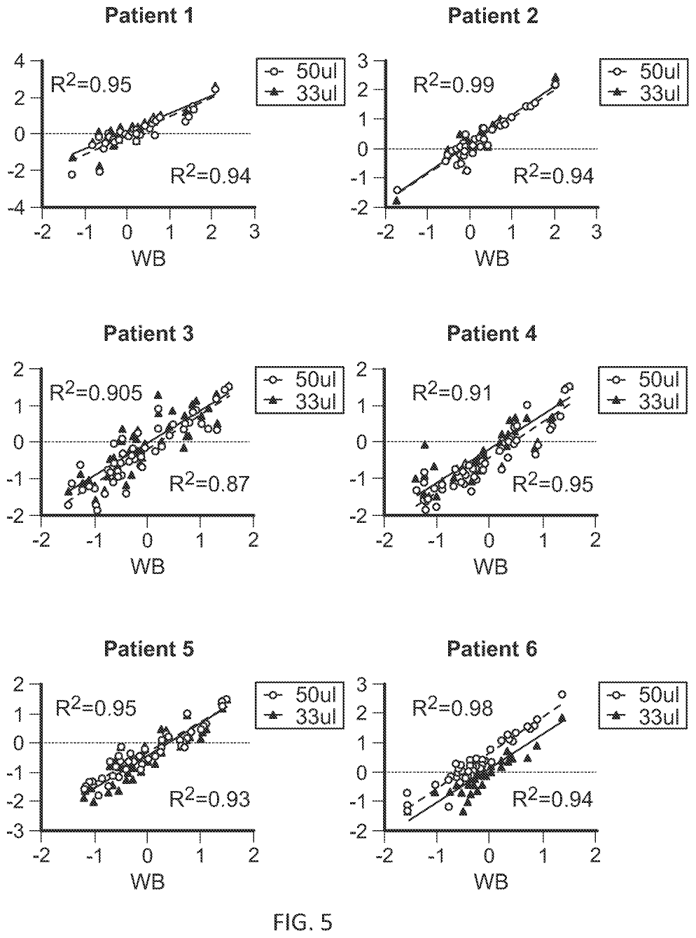

As shown in FIG. 5, regression analyses demonstrate that gene expression is very closely correlated between the stabilized whole blood and stabilized 50 μl fingerprick samples (R²=0.91-0.99, median: 0.95). Correlations were lower for stabilized 35 μl fingerprick samples (R²: 0.87-0.95, median: 0.94).

Levels in stabilized 35 μl fingerprick samples (22±11) were lower and included two samples that were classified as “normal”.

These results indicate that various volumes of collected blood, including volumes ranging from 35 to 50 μl of blood, can be stabilized using the methods and compositions of the present disclosure for use in subsequent diagnostic, prognostic and therapeutic applications.

### Example 4—Venipuncture Blood Stabilized Using Compositions and Methods of the Present Disclosure

Blood was collected from thirteen neuroendocrine tumor patients and 6 normal subjects by venipuncture directly into the stabilization buffer. The stabilized venipuncture blood samples were then incubated at room temperature (21° C.-28° C.) for 0-28 days. The NETest scores at each of the time points was compared to identify how stable the signature was at room temperature.

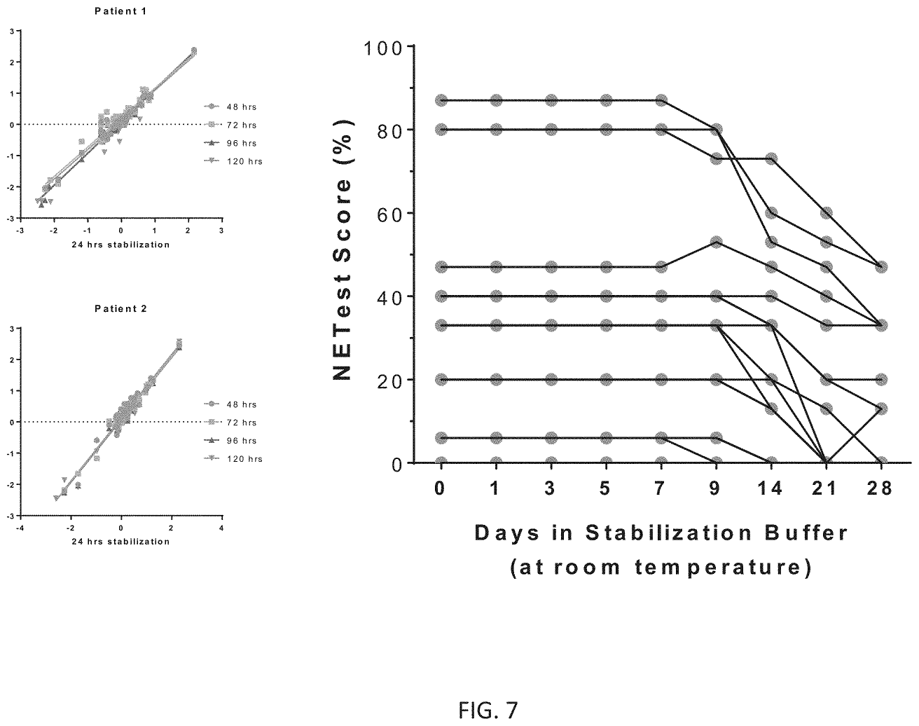

As shown in the left panel of FIG. 7, an assessment of gene expression comparing 2, 3, 4- and 5-days room temperature stabilization with one day room temperature stabilization identified this was significantly concordant in the two cases chosen. Correlations ranged between 0.96-0.99 (p<0.0001) in patient #1 to 0.97-0.985 (p<0.0001) in patient #2.

As shown in the right panel of FIG. 7, NETest scores were 31.1±6.2 at day 0. Scores remained identical up to day 7 (all days: 31.1±6.2). After 9 days in stabilization buffer at room temperature, the score was 30.3±6.1. Thereafter, the score significantly (p<0.0001) decreased to 23.7±5 (day 14), to 15±4.8 (day 21) to 12.6±4 (day 28).

As shown in the right panel of FIG. 7, all patients with high scores (80-100), which is prognostic for disease progression, exhibited stable scores up to day 7. All patients with lower scores (20-50) exhibited stable scores up to day 7. Thus, these results demonstrate that the stabilization methods and compositions of the present disclosure can effectively stabilize a blood sample for at least 7 days for subsequent use in diagnostic, prognostic and therapeutic applications, including the NETest.

### Example 5—Stabilization Buffer Preparation

The following non-limiting example describes methods of preparing the stabilization buffers of the present disclosure.

First, 100 ml of 0.3M EDTA is prepared by:
a. weighing out 8.768 g of EDTA and place in a 250 mL glass beaker;
b. adding about 70 mL water (RNase free) to beaker and stirring with low heat on plate;
c. adding NaOH pellets and measuring with a pH probe until pH begins to reach 7; and
d. when EDTA is visibly dissolved, adding water to the 100 mL mark of the beaker. The final pH for 0.3M should be around 7.5. The EDTA will dissolve before a pH of 7.5. The final pH of the 100 mL solution needs to be between 7.4-7.5
   Then, 1000 ml of the stabilization buffer is prepared by:
a. weighing out 429.88 g of guanidine hydrochloride, 35.3 g of sodium citrate, 15.36 g of citric acid and adding to a clean beaker;
b. adding just enough water (about 500 ml) to the beaker that reagents dissolve and begin stirring on plate;
c. pipetting 66.6 mL of the 0.3M EDTA stock into the beaker;
d. pipetting 1000 μL of Triton X-100 into the beaker using a micropipette. The excess Triton X-100 from the micropipette tip by pipetting up and down with the liquid from the beaker;
e. heating the beaker on the hot plate. Triton will appear as gel-like drops and strands in the liquid. Continue to heat and stir with a stir bar until there is no visible Triton and other solids;
f. adding water to the 1000 mL mark of the beaker;
g. measuring the pH of the buffer with a pH probe. Final pH will be around 4.05.

The pH usually falls between 4.05-4.1.

The stabilization buffer can then be dispensed into a sample tube by injecting 4 ml of final buffer into a 6 ml (16×100 mm) glass or plastic tube that is pre-coated with K2-EDTA (10.8 mg/tube).

### Example 6—RNA Extraction Using TRIzol and the Stabilization Methods and Compositions of the Present Disclosure

The following non-limiting example describes exemplary methods of extracting RNA from biological samples stabilized using the compositions and methods of the present disclosure.

RNA can be extracted from a blood sample stabilized using the stabilization methods and compositions of the present disclosure using the following protocol:
a. Determine the volume of starting material (blood in stabilization buffer). The starting material will typically be 150 μL of stabilized blood. Bring the total volume to 250 μL by adding 100 μL of RNase free water;
b. Add 750 μL of TRIzol LS to the 250 μL of the diluted, stabilized blood sample for a final volume of 1 ml;
c. Vortex the TRIzol LS and sample for 10 seconds and incubate at room temperature for 5 minutes;
d. Add 200 μL of Chloroform per 1 mL of TRIzol/sample and secure the tube cap tightly;
e. Vortex the mixture on high for 15 seconds;
f. Incubate the mixture at room temperature for 3 minutes;
g. Centrifuge the mixture at 13,000×g at 4° C. for 25 minutes;
h. Transfer the aqueous phase (yellow to clear colored layer) to new tubes using a 200 μL pipette;
i. Add 500 μL of Isopropanol to the transferred aqueous phase and then invert the tubes four times;
j. Incubate the mixture at room temperature for 10 minutes;
k. Centrifuge the mixture at 13,000×g at 4° C. for 10 minutes;
l. Pour off the supernatant without disturbing the pellet, gently blotting out any excess supernatant on the edges;
m. Add 1000 μL of 70% ethanol to the pellet;
n. Flick and invert the tubes to float the pellet;
o. Centrifuge the mixture at 10,000×g at 4° C. for 5 minutes;
p. Pour off the supernatant and gently blot out any excess supernatant on the edges;
q. Allow the pellet to air-dry for 10 minutes;
r. Add 100 μL of RNase free water to the pellet and let pellet dissolve for 10 minutes at room temperature.

Proceed to RNA Cleanup Protocol (e.g. using a commercially available kit such as a Qiagen RNeasy Mini Kit).

### Example 6—Saliva Samples and Saliva RNA Stabilized Using the Compositions and Methods of the Present Disclosure

The following non-limiting example demonstrates that the compositions and the methods of the present disclosure can be used to stabilize saliva samples, more specifically the RNA in saliva samples, for extended periods of time at a variety of different temperatures.

In a non-limiting application, the saliva samples and the saliva RNA stabilized using the compositions and methods of the present disclosure can be used as part of a diagnostic test for SARS-CoV-2 infection in a subject. As would be appreciated by the skilled artisan, SARS-CoV-2 is the virus responsible for Coronavirus disease 2019. A non-limiting example of a SARS-CoV-2 is the 3-gene molecular diagnostic test described in the present disclosure. Briefly, the SARS-CoV-2 3-gene molecular diagnostic test measures the expression level of three genes (2 viral target genes and 1 positive control gene) by quantitative PCR using cDNA transcribed from RNA extracted from saliva samples from a subject. The two viral genes are N1 and N3, which encode the viral capsid. The positive control gene can be any positive control nucleic acid known and used in the art, including, but not limited to a specific SARS-CoV-2 control plasmid. Moreover, the expression level of a quality-control gene, such as, but not limited to RNase P, can be measured in order to determine if the saliva sample was appropriately collected and processed.

Saliva samples are first isolated from subjects using swabs. The isolation can take place, for example, in a doctor's office, a remote testing facility, or even at the subject's home. The saliva sample can then be stabilized using the compositions and methods of the present disclosure. At any time after stabilization, RNA can be isolated and purified from the sample using techniques standard in the art (e.g. using the QIAmp Viral RNA mini kit from QIAgen). The purified RNA can then be transcribed into cDNA using techniques that are standard in the art (e.g. using the ThermoFisher High-Capacity cDNA Reverse Transcription Kit). The expression level of the two viral genes and a dilution curve of the positive control gene can then be measured using quantitative PCR using standard techniques that would be appreciated by the skilled artisan. The measured cycle threshold (cT) for the two viral genes can then be compared to cT values in the positive control gene dilution curve to identify a sample as being positive or not for the SARS-CoV-2 virus. The expression level of a quality-control gene, such as RNase P, can also be monitored.

### Example 6a

Saliva samples collected from SARS-CoV-2 positive subjects into a stabilization buffer of the present disclosure comprising guanidine hydrochloride at a concentration of about 4.5 M, Triton X-100 at a concentration of about 0.1% (volume/volume), EDTA at a concentration of about 20 mM, citric acid at a concentration of about 80 mM, sodium citrate at a concentration of about 120 mM, and a pH between about 4.05 and about 4.11.

Each saliva sample was then split into two subsamples.

One of the subsamples was evaluated immediately after sample acquisition as described above. RNA was isolated and purified from the subsample, and the expression level of the diagnostic genes (N1, N3 and RNase P) were measured using quantitative PCR.

The other subsample was allowed to incubate for 5 days (120 hrs) at room temperature (22° C.), prior to RNA isolation and quantitative PCR analysis.

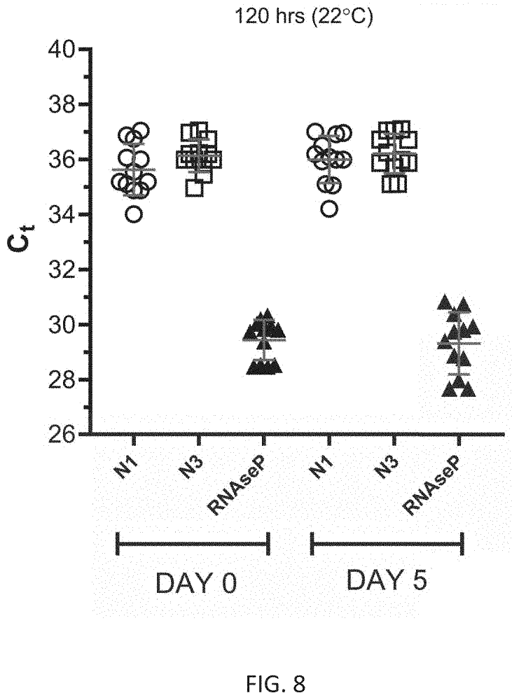

The results of the quantitative PCR analysis of the two subsamples are shown in Table 2 and in FIG. 8. The subsamples that were analyzed immediately following sample acquisition are referred to in Table 2 and FIG. 8 as “Day 0” samples, and the subsamples that were allowed to incubate for 5 days at room temperature are referred to as “Day 5” samples.

TABLE 2

| SARS-CoV-2 | Number of | Detection | N1 C_{T} | N3 C_{T} | RNAseP C_{T} |
|---|---|---|---|---|---|
| concentration | samples | rate | (mean) | (mean) | (mean) |
| Day 0 | 12 | 12 | 35.63 | 36.14 | 29.44 |
| Day 5 | 12 | 12 | 36.00 | 36.21 | 29.31 |
| p-value | | | 0.13 | 0.73 | 0.64 |
| (wilcoxan | | | | | |
| matched-pairs | | | | | |
| signed rank | | | | | |
| test) | | | | | |

As shown in Table 2 and in FIG. 8, the measured expression levels (Ct values) for each of the genes were approximately the same in the Day 0 subsamples and the day 5 subsamples. Moreover, all 12 samples were accurately diagnosed as SARS-CoV-2 positive. Without wishing to be bound by theory, these results indicate that the stabilization buffer of the present disclosure stabilized the saliva sample such that the RNA was protected from degradation over the course of the 5-day, room temperature incubation.

### Example 6b

Saliva samples collected from subjects into a stabilization buffer of the present disclosure comprising guanidine hydrochloride at a concentration of about 4.5 M, Triton X-100 at a concentration of about 0.1% (volume/volume), EDTA at a concentration of about 20 mM, citric acid at a concentration of about 80 mM, sodium citrate at a concentration of about 120 mM, and a pH between about 4.05 and about 4.11.

Each saliva sample was then split into two subsamples.

One of the subsamples was evaluated immediately after sample acquisition as described above. RNA was isolated and purified from the subsample, and the expression level of the diagnostic genes (N1, N3 and RNase P) were measured using quantitative PCR.

The other subsample was subjected to thermal excursion at temperatures ranging from 22 to 40° C. for 56 hours according to the profile presented in Table 3 prior to RNA isolation and quantitative PCR analysis.

TABLE 3

| Cycle | | Cycle | Total Time |
|---|---|---|---|
| Period | Temperature | Period (Hours) | Elapsed (Hours) |
| 1 | 40° C. | 8 | 8 |
| 2 | 22° C. | 4 | 12 |
| 3 | 40° C. | 2 | 14 |
| 4 | 30° C. | 36 | 50 |
| 5 | 40° C. | 6 | 56 |

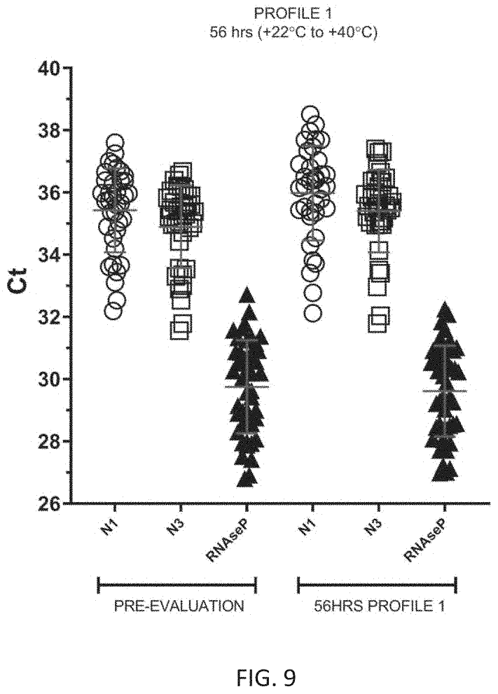

The results of the quantitative PCR analysis of the two subsamples are shown in Table 4 and in FIG. 9. The subsamples that were analyzed immediately following sample acquisition are referred to in Table 4 and FIG. 9 as “Pre-evaluation” samples, and the subsamples that were subjected to the 56-hour thermal excursion are referred to as “56 Hrs Profile 1” samples.

TABLE 4

| SARS-CoV-2 | Number of | Detection | N1 C_{T} | N3 C_{T} | RNAseP C_{T} |
|---|---|---|---|---|---|
| concentration | samples | rate | (mean) | (mean) | (mean) |
| Pre-evaluation | 48 | 38/38+ | 35.43 | 34.89 | 29.75 |
| | | 0/10− | | | |
| 56 Hrs | 48 | 37/38+ | 35.98 | 35.40 | 29.61 |
| Profile 1 | | 0/10− | | | |
| p-value* | | | 0.08 | 0.09 | 0.71 |
| *Wilcoxan | | | | | |
| matched-pairs | | | | | |
| signed rank | | | | | |
| test | | | | | |

As shown in Table 4 and in FIG. 9, the measured expression levels (Ct values) for each of the genes were approximately the same in the Pre-evaluation subsamples and the day 56 Hrs Profile 1 subsamples. Moreover, Thirty-seven of 38 (97%) spike-in samples were detected in 56 Hrs Profile 1 subsamples. Twenty-two of 23 (96%) of low viral titer samples were accurately, detected, and all higher viral titer samples were detected. All controls (no spike-in) were negative for SARS-CoV-2 detection. Without wishing to be bound by theory, these results indicate that the stabilization buffer of the present disclosure stabilized the saliva sample such that the RNA was protected from degradation over the course of a 56-hour temperature excursion, with temperatures ranging from 22 to 40° C.

### Example 6c

Saliva samples collected from subjects into a stabilization buffer of the present disclosure comprising guanidine hydrochloride at a concentration of about 4.5 M, Triton X-100 at a concentration of about 0.1% (volume/volume), EDTA at a concentration of about 20 mM, citric acid at a concentration of about 80 mM, sodium citrate at a concentration of about 120 mM, and a pH between about 4.05 and about 4.11.

Each saliva sample was then split into two subsamples.

One of the subsamples was evaluated immediately after sample acquisition as described above. RNA was isolated and purified from the subsample, and the expression level of the diagnostic genes (N1, N3 and RNase P) were measured using quantitative PCR.

The other subsample was subjected to thermal excursion at temperatures ranging from −10 to 18° C. for 56 hours according to the profile presented in Table 5 prior to RNA isolation and quantitative PCR analysis.

TABLE 5

| Cycle | | | Cycle | Total Time |
|---|---|---|---|---|
| Period | Temperature | | Period (Hours) | Elapsed (Hours) |
| 1 | −10° | C. | 8 | 8 |
| 2 | 18° | C. | 4 | 12 |
| 3 | −10° | C. | 2 | 14 |
| 4 | 10° | C. | 36 | 50 |
| 5 | −10° | C. | 6 | 56 |

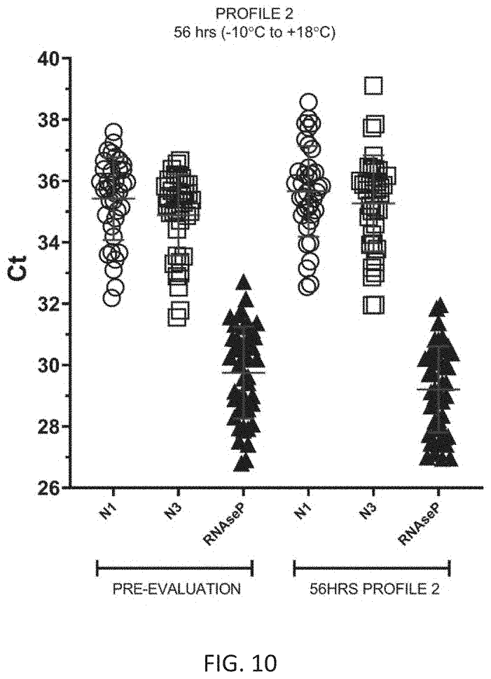

The results of the quantitative PCR analysis of the two subsamples are shown in Table 6 and in FIG. 10. The subsamples that were analyzed immediately following sample acquisition are referred to in Table 6 and FIG. 10 as “Pre-evaluation” samples, and the subsamples that were subjected to the 56-hour thermal excursion are referred to as “56 Hrs Profile 2” samples.

TABLE 6

| SARS-CoV-2 | Number of | Detection | N1 C_{T} | N3 C_{T} | RNAseP C_{T} |
|---|---|---|---|---|---|
| concentration | samples | rate | (mean) | (mean) | (mean) |
| Pre-evaluation | 48 | 38/38+ | 35.43 | 34.89 | 29.75 |
| | | 0/10− | | | |
| 56 Hrs | 48 | 38/38+ | 35.67 | 35.27 | 29.21 |
| Profile 2 | | 0/10− | | | |
| p-value* | | | 0.75 | 0.23 | 0.08 |
| *Wilcoxan | | | | | |
| matched-pairs | | | | | |
| signed rank | | | | | |
| test | | | | | |

As shown in Table 6 and in FIG. 10, the measured expression levels (Ct values) for each of the genes were approximately the same in the Pre-evaluation subsamples and the day 56 Hrs Profile 1 subsamples. Moreover, all spike-in samples were detected (38/38, 100%) and all controls (10/10 no spike-in) were negative for SARS-CoV-2 detection. Without wishing to be bound by theory, these results indicate that the stabilization buffer of the present disclosure stabilized the saliva sample such that the RNA was protected from degradation over the course of a 56-hour temperature excursion, with temperatures ranging from −10 to 18° C.

1. A stabilization buffer comprising:
(a) guanidine hydrochloride at a concentration of about 4.05 M to about 4.95 M;
(b) Triton X-100 at a concentration of about 0.09% to about 0.11% (v/v); and
(c) EDTA at a concentration of about 18 mM to about 22 mM,
wherein the pH of the stabilization buffer is less than about 4.5.

2. The stabilization buffer of claim 1, wherein the stabilization buffer comprises:
(a) guanidine hydrochloride at a concentration of about 4.275 M to about 4.725 M;
(b) Triton X-100 at a concentration of about 0.095% to about 0.105% (v/v); and
(c) EDTA at a concentration of about 19 mM to about 21 mM.

3. The stabilization buffer of claim 2, wherein the stabilization buffer comprises:
(a) guanidine hydrochloride at a concentration of about 4.5 M;
(b) Triton X-100 at a concentration of about 0.1% (volume/volume); and
(c) EDTA at a concentration of about 20 mM.

4. The stabilization buffer of claim 1, wherein the pH of the stabilization buffer is between about 4.05 and about 4.11.

5. The stabilization buffer of claim 1, further comprising:
(d) citric acid at a concentration of about 72 mM to about 88 mM; and
(e) sodium citrate at a concentration of about 108 mM to about 132 mM.

6. (canceled)

7. The stabilization buffer of claim 5, wherein the stabilization buffer comprises:
(d) citric acid at a concentration of 80 mM; and
(e) sodium citrate at a concentration of 120 mM.

8. The stabilization buffer of claim 1, wherein the stabilization buffer comprises:
(a) guanidine hydrochloride at a concentration of about 4.5 NT;
(b) Triton X-100 at a concentration of about 0.1% (volume/volume),
(c) EDTA at a concentration of about 20 mM;
(d) citric acid at a concentration of 80 mM; and
(e) sodium citrate at a concentration of 120 mM,
wherein the pH of the stabilization buffer is between about 4.05 and about 4.11.

9. A composition comprising a mixture of:
(a) the stabilization buffer of claim 1; and
(b) a biological sample isolated from a subject.

10. The composition of claim 9, wherein the biological sample comprises blood, plasma, serum, urine, breast milk, cerebrospinal fluid, mucus, gastric juice, peritoneal fluid, pleural fluid, saliva, sebum, semen, sweat, tears, vaginal secretion, vomit, endolymph, perilymph or any combination thereof.

11. The composition of claim 9, wherein the biological sample is a blood sample.

12. The composition of claim 10, wherein the biological sample is a saliva sample.

13. The composition of claim 9, wherein the biological sample comprises at least one RNA transcript, wherein the amount of the at least one RNA transcript decreases by no more than about 5% after the composition is incubated for at least one day at room temperature.

14. A kit comprising the stabilization buffer of claim 1.

15. The kit of claim 14, comprising the stabilization buffer in at least one sample collection tube, wherein the at last one sample collection tube is pre-coated with K2-EDTA.

16. The kit of claim 15, wherein the at least one sample collection tube is a 6 ml, 16×100 mm sample collection tube, wherein the at least one sample tube is pre-coated with at least about 10.8 mg of K2-EDTA.

17. A method of stabilizing a biological sample from a subject, the method comprising contacting the biological sample and the stabilization buffer of claim 1, thereby producing a stabilized biological sample.

18. The method of claim 17, wherein the biological sample comprises blood, plasma, serum, urine, breast milk, cerebrospinal fluid, mucus, gastric juice, peritoneal fluid, pleural fluid, saliva, sebum, semen, sweat, tears, vaginal secretion, vomit, endolymph, perilymph or any combination thereof.

19. The method of claim 18, wherein the biological sample is a blood sample.

20. The method of claim 18, wherein the biological sample is a saliva sample.

21. The method of claim 17, wherein
the biological sample comprises at least one RNA transcript, wherein the expression level of the at least one RNA transcript in the stabilized biological sample measured after incubating the stabilized biological sample for at least 24 hours at about room temperature is within about 5% (±5%).

22. The method of claim 21, wherein the expression level of the at least one RNA transcript is measured using quantitative PCR.

23. The method of claim 17, the method further comprising:
i) extracting RNA from the stabilized biological sample;
ii) determining the expression level of at least one RNA transcript in the extracted RNA; and
iii) administering at least one therapeutic to the subject based on the expression level of the at least one RNA transcript.

24. The method of claim 23, wherein determining the expression level of the at least one RNA transcript in the extracted RNA comprises the use of quantitative PCR.

25. (canceled)

26. The method of claim 23, wherein the subject has cancer, gastroenteropancreatic (GEP) neuroendocrine neoplasm (GEP-NEN), melanoma, multiple myeloma, a plasma cell dyscrasia, monoclonal gammopathy of undetermined significance (MGUS), colon cancer, prostate cancer or SARS-CoV-2 infection.

27. (canceled)
